(19) United States
(12) Patent Application Publication
(10) Pub. No.: US 2004/0191786 A1
(43) Pub. Date: Sep. 30, 2004

(54) Three cube fret method (3-fret) for detecting fluorescence energy transfer

(76) Inventors: David T. Yue, Baltimore, MD (US); Michael G. Erickson, Baltimore, MD (US)

Correspondence Address: Lisa Swiszcz Hazzard, Edwards & Angell, PO Box 9169, Boston, MA, 02209, US

(21) Appl. No.: 10/467,964

(22) PCT Filed: Feb. 15, 2002
(86) PCT No.: PCT/US02/04563

Related U.S. Application Data
(60) Provisional application No. 60/269,669, filed on Feb. 16, 2001.

(51) Int. Cl. C12Q 1/68; G01N 33/53; G06F 19/00; G01N 33/48; G01N 33/50

(52) U.S. Cl. 435/6; 435/7.1; 702/20

(57) ABSTRACT

The invention provides a method for determining a measure of FRET comprising obtaining sequential fluorescent intensity readings from a specimen, such as a cell using three filter cubes. Simple equations manipulate readings from each of the filter sets or cubes to specify a unitless index of FRET called the FRET ratio (FR). FR bears a linear relation to FRET efficiency E. The method also provides for determining the fraction of acceptor-tagged molecules bound by donor-tagged molecules; the relative affinity of binding; and the strength of FRET interactions when all acceptor-tagged molecules are bound by donor. The latter determination enables estimates of the physical distance and/or orientation between interacting fluorophore molecules. The method can be used to detect analytes or inter- or intramolecular interactions. In a preferred aspect, the method is used in an HTS assay to identify modulators of such interactions.

### RELATED APPLICATIONS

This application claims priority under 35 U.S.C. § 119(e) to U.S. Provisional Application No. 60/269,669, filed Feb. 16, 2001, and to U.S. Provisional Application No. 60/275,911, filed Mar. 15, 2001, the entireties of which are incorporated by reference herein.

### FIELD OF THE INVENTION

The invention relates to methods for detecting and quantifying Fluorescent Resonance Energy Transfer (FRET). In particular, the invention, termed the 3³-FRET method, furnishes the means to perform quantitative, FRET-based assays for measuring inter- or intramolecular interactions, in a manner that is especially suited to the conditions encountered in living cells.

### BACKGROUND OF THE INVENTION

High throughput screening (HTS) assays for compounds that alter either inter- or intra-molecular interactions are widely used to screen large numbers of test compounds for potential therapeutic activity. Methods for monitoring cellular responses of a drug target (e.g., such as an extracellular receptor) to a test compound using optically detectable labels can provide a sensitive and quantitative measure of the target's activity. In addition to providing platforms for identifying new drugs, cell-based assays also can be used to characterize the physiological function of a target biomolecule, for example, by identifying changes in a target biomolecule's function in response to physiological stimuli. Functional assays can range from binding assays (e.g., library-based screening methods) to genetic assays (e.g., screens for extragenic suppressors or activators) (see, e.g., Phizicky and Fields, 1995, Microbiol. Rev. 59: 94-123).

One technique for assessing intermolecular interactions is based on fluorescence resonance energy transfer (FRET) (see Selvin, 1995, Methods Enzymol. 246: 300-334). In this process, a “donor” fluorophore transfers its excited-state energy to an “acceptor” fluorophore which typically emits fluorescence of a different color. Suitable donor and acceptor fluorophore pairs are those that exhibit substantial overlap between respective emission and excitation spectra (Selvin, 1995, Methods Enzymol. 246: 300-334). FRET has been used in both in vitro and in vivo assays to monitor protein-protein interactions by chemically attaching appropriate fluorophores to pairs of purified proteins and measuring fluorescence spectra of protein mixtures or cells microinjected with the labeled proteins (see, e.g., Adams, et al., 1991, Nature 349: 694-697).

The cloning and expression of spontaneously fluorescent proteins has facilitated genetic labeling of proteins with fluorophores. One prominent example is green fluorescent protein (GFP) from the jellyfish, Aequorea victoria. The cDNA encoding GFP can be fused with coding sequences from a number of other proteins, thus enabling such proteins to fluoresce without interfering with their biological activity or cellular localization. Further, mutant variants of spontaneously fluorescent proteins with different emission wavelengths across the visible spectrum provide a variety of suitable donor:acceptor pairs for FRET (see, e.g., Heim, et al., 1994, Proc. Nat. Acad. Sci. U.S.A. 91: 12501-12504). For example, enhanced cyan fluorescent protein (ECFP) and enhanced yellow fluorescent protein (EYFP) are color variants of GFP that are suitable for FRET applications, and this donor:acceptor pair has been used in vivo to monitor changes in protein conformation (see, e.g., Miyawaki, et al., 1997, Nature 388: 882-887).

Even with the engineering of novel genetically-encoded fluorophores, measurement of FRET in living cells entails several challenges. Variability in expression levels and fractional binding of acceptor- and donor-tagged molecules are inevitable in live cells and complicate quantitation of the strength of FRET. Inability to selectively excite donor fluorophores, as well as inability to selectively detect acceptor emission, are often experienced with many FRET pairs, including the ECFP/EYFP pair. These “crosstalk” constraints further complicate quantitation of FRET. In practice, detection of FRET in living cells can be difficult, destructive of the sample, and/or time-consuming. The challenges of incomplete labelling, variable concentrations of fluorophore, and variable fractional binding between fluorophore-tagged molecules may extend beyond the setting of genetically-encoded fluorophores.

### SUMMARY OF THE INVENTION

The invention (3³-FRET) provides a fast, simple, and nondestructive method for detecting and quantifying FRET, despite the aforementioned challenges. One advantage of the 3³-FRET method is that it provides a way to nondestructively determine a quantitative index of the strength of FRET interactions, despite variable expression levels and variable bound fractions of acceptor- and donor-tagged molecules. The specific index of FRET is termed “the FRET ratio,” or FR. A second advantage of the 3³-FRET method is that it provides a way to nondestructively determine: the fraction of acceptor-tagged molecules that are bound by donor-tagged molecules; the relative affinity of a binding reaction; and the strength of FRET interactions when all acceptor-tagged molecules are bound by donor-tagged molecules. The latter determination enables estimates of the physical distance and/or orientation between interacting acceptor and donor fluorophore molecules to be obtained. This second advantage may be conveniently applied to determinations of FR, but may also be applied to many other quantitative FRET indices.

In one aspect, the invention provides a method for detecting interactions between two molecules or between different portions of a single molecule. The method comprises processing measurements made from a specimen containing donor and acceptor fluorophores, which are attached to either separate molecules or different parts of the same molecule. The specimen is exposed to a wavelength of light suitable for exciting donor molecules and the light emitted by the specimen is detected and decomposed to determine whether acceptor molecules have received energy from donor molecules, i.e., indicating the relative proximity of the donor and acceptor molecules.

To accomplish this decomposition, three filter sets are sequentially placed between the light source and the specimen, and between the specimen and the detector (FIG. 8A). The individual filter sets each comprise a filter between the light source and the specimen and a filter between the specimen and the detector. Each filter set transmits and/or reflects specific wavelengths of light. In the first filter set (“donor filter set”), the filter between the light source and specimen maximally transmits a wavelength of light that excites the donor (and possibly the acceptor), and the filter between the specimen and the detector maximally transmits wavelengths of light where only the donor emits photons. In the second filter set (“acceptor filter set”), the filter between the light source and specimen maximally transmits a wavelength of light that preferentially excites the acceptor, and the filter between the specimen and the detector maximally transmits wavelengths of light where mainly the acceptor emits photons (and possibly the donor emits photons). In the third filter set (“FRET filter set”), the filter between the light source and specimen maximally transmits a wavelength of light that excites the donor (and possibly the acceptor), and the filter between the specimen and the detector maximally transmits wavelengths of light where mainly the acceptor emits photons (and possibly the donor emits photons).

The 3³-FRET method processes these three light intensity readings, each obtained with a different filter set engaged, and yields a quantitative readout of the strength of FRET interaction, termed “the FRET ratio” or FR. FR furnishes the fractional increase in acceptor fluorescence due to FRET.

Preferably, three filter cubes comprise the first, second, and third filter sets. Preferably, each filter cube contains an excitation filter, a dichroic mirror, and an emission filter.

In one aspect, the donor molecule is a polypeptide such as ECFP and the acceptor molecule is a polypeptide such as EYFP. Preferably, the FRET ratio is produced by processsing sequential filter set measurements according to:

$$FR = \frac{[ {S}_{FRET} ( DA ) - {R}_{D1} \cdot {S}_{D} ( DA ) ]}{{R}_{A1} \cdot [ {S}_{A} ( DA ) - {R}_{D2} \cdot {S}_{D} ( DA ) ]}$$

wherein S_{FRET}(DA) is a measure of light intensity transmitted to the detector from the FRET filter set, S_{D}(DA) is a measure of light intensity transmitted to the detector from donor filter set, and S_{A}(DA) is a measure of light intensity transmitted to the detector from the acceptor filter set. R_{D1}, R_{A1}, and R_{D2} are predetermined constants determined from measurements of light emissions from specimens expressing only donor (D) or acceptor (A) molecules (see Equations A6-A8 in Detailed Description, below). In practice, no two optical systems are identical; for example, small aberrations in optical components comprising the filter sets are common. Because FR is unitless, this index of FRET has the special advantage of being independent of these small aberrations; all errors of this sort are “normalized out” in producing this ratio.

In one aspect, the method comprises processing like measurements from multiple specimens, and furnishing an estimate of the relative affinity of the binding of donor-tagged molecules to acceptor-tagged molecules, the fractional binding of acceptor-tagged molecules by donor-tagged molecules in any individual specimen, and the maximum FRET efficiency when every acceptor-tagged molecule is associated with a donor-tagged molecule.

In another aspect, the method comprises providing an estimate of the relative affinity of the binding of acceptor-tagged molecules to donor-tagged molecules, the fractional binding of donor-tagged molecules by acceptor-tagged molecules in any individual specimen, and the maximum FRET efficiency when every donor-tagged molecule is associated with a acceptor-tagged molecule. These last two aspects of the invention (summarized in FIG. 8B) are conveniently applied to determinations of FR, but may also utilize many other quantitative FRET indices.

The maximum FRET efficiency can be used to determine the physical distance and/or orientation between donor and acceptor molecules. In one aspect, the maximum FRET efficiency can be gauged by FR^{max}, the maximum FRET ratio when every acceptor-tagged molecule is associated with a donor-tagged molecule. The classic index of FRET efficiency, termed E, can then be produced by processing FRₘₐₓ according to:

E=(FRₘₐₓ−1)[ε_{A}(λex)/ε_{D}(λex)],

wherein the bracketed term is the ratio of acceptor and donor molar extinction coefficients at the preferred wavelength of the filter between the light source and specimen in the FRET filter set. Assuming randomized orientation of donor and acceptor transition dipoles during the time course of FRET interactions, donor:acceptor distance then can be determined according to:

R=R₀(E⁻¹−1)^{1/6}, wherein R₀=49

In one aspect, the specimen is a cell and the method further comprises the step of introducing the donor and acceptor molecule into the cell. For example, the donor and acceptor molecule can be introduced by transfection (e.g., cDNA transfection), transformation, electroporation, microinjection, or a combination thereof. The donor and acceptor molecule can each be linked to different biomolecules, using standard molecular biological techniques. In one aspect, the different biomolecules are binding partners, e.g., interacting polypeptides, nucleic acids, or nucleic acids and nucleic acid binding proteins. In another aspect, one of the polypeptides is selected from the group consisting of calmodulin (CaM), cGMP-dependent protein kinase, a steroid hormone receptor or a ligand binding domain thereof, protein kinase C, inositol-1,4,5-triphosphate receptor, alphachymotrypsin, or recoverin. One or both of the polypeptides can contain an intracellular localization signal for specific targeting of one or both of the polypeptides within a cell. Detection of FRET can be used to assay for intermolecular interactions in this system.

In one aspect, the cell is exposed to a sample suspected of comprising a modulator of the binding partners and the measure of FRET provides an indication of whether or not the sample comprises the modulator. Preferably, one of the binding partners is an intracellular signaling molecule. Suitable binding partners include, but are not limited to: a ligand and receptor; antibodies and antigens; calmodulin and ion channels; G-proteins and ion channels; and GTP and G-protein coupled receptors.

In another aspect, the method is used to identify interacting molecules (e.g., such as those involved in intracellular signaling processes). For example, the donor molecule is linked to a “bait” polypeptide (e.g., encoding a polypeptide being evaluated such as an orphan receptor), while the acceptor molecule is linked to a “prey” polypeptide (e.g., an unknown polypeptide sequence taken from a library or expressed sequences such as a cDNA library). The measure of FRET provides a measure of whether the bait polypeptide and prey polypeptide specifically bind to each other. Single-cell purification of plasmid DNA (“single-cell miniprep”) can be used to specify the sequence identity of nucleic acids encoding the interacting prey polypeptide. In this manner, discovery of unknown interaction partners with a specified bait polypeptide can be determined. For example, the assay can be used to identify ligands for orphan receptors. Application of this approach to many cells in parallel, such as using plate-reader technology, permits high-throughput identification of interacting molecules. The assay also can be used to identify interacting molecules in living mammalian cells.

In one aspect, the method can be used to identify mutations or compounds that inhibit and/or promote binding between two molecules known to interact. For example, mutations can be introduced into polypeptides fused to either donor or acceptor fluorophores. Loss or enhancement of FRET interaction between binding partners indicates a critical site for interaction was mutated. As another example, cells expressing interacting FRET partners can be exposed to a library of compounds Loss and/or enhancement of FRET indicate a compound that may modulate the interaction between specific FRET-pair molecules. Because the 3³-FRET method is nondestructive, time-dependent aspects of compound modulation may be examined. Application of this approach to many cells in parallel, such as using plate-reader technology, permits high-throughput identification of important mutations or modulatory molecules.

The donor and acceptor molecule also can be linked to a single molecule (e.g., a nucleic acid or polypeptide) for detecting an analyte. In one aspect, the molecule for detecting an analyte specifically binds to the analyte. In another aspect, the molecule for detecting an analyte is cleavable by the analyte. For example, the molecule for detecting an analyte may comprise a polypeptide comprising a protease cleavage site or may comprise a nucleic acid comprising a nuclease digestion site. In a further aspect, the molecule for detecting an analyte is immobilized on a solid phase, thereby forming a FRET sensor. The FRET sensor can be exposed to a sample suspected of comprising the analyte, and the measure of FRET obtained can be correlated with the presence or level of the analyte.

The donor molecule and acceptor molecule linked to a single molecule for detecting an analyte also can be introduced into a cell and the measure of FRET can be correlated with the presence or level of analyte in the cell.

In one aspect, the method further comprises the step of sorting cells comprising donor and acceptor molecules from those which do not comprise both donor and acceptor molecules. In another aspect, the method comprises the further step of sorting cells in which FRET occurs from cells in which FRET does not occur.

An optical system can be used to perform the methods described above and in one aspect, the optical system comprises a light source for providing excitation light to the specimen; the detector; a specimen holder for positioning the specimen in a suitable position to receive light from the light source sufficient to excite the donor, and to transmit light emitted by the cell to the detector; and a bolder for sequentially receiving the first, second, and third filter sets, and for positioning each of the filters. Preferably, the optical system is selected from the group consisting of an epifluorescence microscope, a confocal microscope, a flow cytometer, and a plate reader.

In summary, the 3³-FRET invention provides a fast, simple, and nondestructive method for detecting and quantifying FRET. One main part of the 3³-FRET method provides means to sensitively and selectively produce a quantitative index of the strength of FRET interaction. The process controls for variability in expression levels and fractional binding of acceptor- and donor-tagged molecules; for inevitable small aberrations in optical components used to perform FRET measurements; and for optical crosstalk between acceptor and donor fluorophores. A advantage of the 3³-FRET method provides a means to determine: the fraction of acceptor-tagged molecules that are bound by donor-tagged molecules; the relative affinity of that binding reaction; and the strength of FRET interaction when all acceptor-tagged molecules are bound by donor-tagged molecules. The latter determination enables estimates of the physical distance and/or orientation between interacting acceptor and donor fluorophore molecules.

### BRIEF DESCRIPTION OF THE FIGURES

The objects and features of the invention can be better understood with reference to the following detailed description and accompanying drawings.

FIGS. 1A-F show that CaM_{WT}-ECFP and α_{1C}-EYFP preserve Ca²⁺-dependent inactivation. FIG. 1A shows the β₂ₐ subunit and CI region (Peterson, et al., 1999, Neuron 22: 549-558) of α_{1C}-EYFP. FIG. 1B shows a confocal image and intensity profile for a cell expressing α_{1C}-EYFP/β₂ₐ/α₂δ. Peaks indicate membrane targeting. FIG. 1C shows HEK293 lysates probed with anti-CaM or anti-GFP (labelled). Upper left: comparison of control (mock transfected) cells with cells overexpressing CaM_{WT}-ECFP or CaM_{MUT}-ECFP; arrowhead indicates endogenous CaM at ˜20 kD. Lower left: same lysates as above, optimized for visualization of endogenous CaM, showing that endogenous CaM expression is unchanged. Lower right: calibration ladder for purified recombinant CaM_{WT} and CaM_{MUT}, conditions same as at left. Upper right: immunoblot probed with anti-GFP antibody comparing CMV and SV40 promoter systems. FIG. 1D shows whole-cell currents from cells co-expressing α_{1C}-EYFP/β₂ₐ/α₂δ and CaM_{WT}-ECFP. The upper graph shows Ba²⁺ (black) and scaled Ca²⁺ (gray) currents during steps to −10 mV. The lower graph shows the fraction of current remaining at the end of 300 ms depolarizations (r₃₀₀). FIG. 1E shows results from cells co-expressing α_{1C}-EYFP/β₂ₐ/α₂δ and CaM_{MUT}-ECFP using a format identical to FIG. 1D. FIG. 1F shows confocal images and intensity profiles for cells expressing CaM_{WT}-EYFP alone (left) or together with α_{1C}/β₂ₐ/α_{2bδ} (right) showing some perimembrane enrichment of CaM_{WT}-EYFP (peaks in intensity profile) when coexpressed with unlabeled channels.

FIG. 2 illustrates FRET detection by 3³-FRET. FIG. 2A shows dissection of 535 nm emission with 440 nm excitation. The graph shows the overall emission spectrum from a single cell expressing ECFP- and EYFP-tagged proteins (black line), reflecting underlying ECFP (thick gray) and EYFP (thin gray) spectra. Portions of the EYFP emission are due to direct excitation (gray dashed spectra). Points (1-5) are: S_{FRET}(DA); R_{D1}S_{CFP}(DA); S_{FRET}(DA)-R_{D1}S_{CFP}(DA); R_{A1}S_{YFP}(DA); and, S_{CFP}(DA); where R_{D1} and R_{A1} are pre-computed constants from cells expressing only ECFP- or EYFP-tagged proteins, respectively, and are described further in the text below. FIG. 2B shows 3³-FRET control experiments on single live cells expressing indicated constructs. Horizontal axes correspond to the FRET Ratio (FR) and FRET percent efficiency (E). For yellow cameleon-2 constructs, cells were incubated in 10 μM ionomycin for 15 minutes before application of either 5 mM EGTA or 20 mM CaCl in buffered Tyrode's.

FIGS. 3A-B show preassociation of CaM with L-type Ca²⁺ channel complexes. Horizontal axes correspond to the FRET Ratio (FR) and FRET percent efficiency (E); α_{2b}δ subunits also are transfected. As shown in FIG. 3A, 3³-FRET reveals that CaM_{WT}and CAM_{MUT} preassociate with L-type channels in resting cells. Asterisk, p<0.01 vs. free ECFP; dagger, p<0.05. FIG. 3B shows that preassociation with L-type channel complexes requires the α_{1c} pore-forming subunit. dagger, p<0.05

FIG. 4 shows preassociation of CaM with R-Type and P/Q Type Ca²⁺ channel complexes. Format Identical to FIG. 3; α_{2b}δ subunits also are transfected. Asterisk, p<0.01 vs. free ECFP

FIG. 5 shows a model of CaM preassociation. FIG. 5A shows analysis of FR data for cells coexpressing CaM_{WT}-ECFP and α_{1C}-EYFP/β₂ₐ/α_{2b}δ. The upper panels show a comparison of measured (filled circles) and predicted (black line) FR values for cells coexpressing FRET between pairings plotted versus calculated fraction bound, A_{b} Arrowhead indicates the maximal FR, FRₘₐₓ. In addition to using 3³-FRET, FRET also was measured by swapping ECFP and EYFP and quantitating ECFP dequenching following complete acceptor photodestruction (open circles). The center set of panels show the probability distribution function of relative number of molecules, P(N)=Prob{number of molecules N}. N_{D} (Black) and N_{A} (gray) are relative numbers of ECFP-and EYFP-tagged molecules, respectively, as determined using α_{2b}δ. The lower set of panels show the probability distribution function of the ration of ECFP-tagged molecules to EYFP molecules, P(R)=Prob{ratio of ECFP-tagged molecules to EYFP-tagged molecules R}. FIG. 5B shows FR data for cells coexpressing ECFP and α_{1C}-EYFP/β₂ₐ/α_{2b}δ using a format analogous to FIG. 5A. FR-A_{b} data is plotted as mean±SD for visual clarity. FIG. 5C shows FR data for cells expressing yellow cameleon-2 (YC2) in the Ca²⁺ free state. The format is analogous to that of FIG. 5A. FR-A_{b} data is plotted as mean±SD for visual clarity. FIG. 5D shows a table of K_{d,EFF} and FRₘₐₓ values from fits of measured FR. FIG. 5E show FR data for cells coexpressing CaM_{WT}-ECFP and β_{2A}-EYFP (left) or β_{2A}-ECFP and α_{1C}-EYFP/α_{2b}δ. The format is identical to the upper panel of FIG. 5A. FIG. 5F shows course triangulation of key channel landmarks using 3³-FRET analysis. ECFP and EYFP are not represented.

FIG. 6 shows fluorescence behavior of donor (ECFP) and acceptor (EYFP) molecules in a microscope field of view, represented quantitatively as three sequential subsystems: an excitation subsystem, afluorophore-rate-constant subsystem, and emission-detection subsystem. The three output signals on the right are those that comprise aggregate fluorescence output obtained with any of the filter filter sets or cubes used an optical system according to the invention.

FIGS. 7A-C show application of 3³-FRET to two-hybrid screening of Ca²⁺ channel/CaM interactions. FIG. 7A shows examplar “prey” segments from the α_{1C} CI region, and the relevant “bait.” EF, PreIQ and IQ are ˜33-residue domains. FIG. 7B, left, shows screen results for the labelled prey-bait pair, showing that PreIQ, IQ and PreIQ-IQ each interact with CaM_{MUT}. Right, preliminary fits using 1:1 binding model as in FIG. 5. Based on estimates of k_{d,EFF}, the combined PreIQ-IQ segment supports the tightest binding with CaM_{MUT}, suggesting that PreIQ and IQ each contribute to form a high-affinity apoCaM binding pocket. Arrowheads indicate FRₘₐₓ estimates. FIG. 7C shows Ca²⁺-dependent movements in CaM binding to segments of the Ca²⁺ channel (same format as in FIG. 7B). Cells were clamped to either high (10 mM, gray bars) or low (5 mM EGTA, gray bars) Ca²⁺ following 15 minute incubation in ionomycin, a potent Ca²⁺-ionophore. The Ca²⁺-induced increase in FRₘₐₓ, (right, compare black and gray arrowheads) reports a significant conformational change in the prey-bait complex. The reported k_{d,EFF} estimates correspond to the fits for cells clamped at high internal Ca²⁺.

FIG. 8A shows a flow-chart depicting the major steps of the 3³-FRET method for producing FR, the quantitative index of FRET according to one aspect of the invention. FIG. 8B shows a flow-chart depicting the major steps of the 3³-FRET method for producing K_{d,EFF} and FRₘₐₓ.

### DETAILED DESCRIPTION

The invention (3³-FRET) provides a fast, simple, and nondestructive method for detecting and quantifying FRET. The 3³-FRET method can be used to sensitively and selectively determine a quantitative index of the strength of FRET interaction, based on a series of fluorescent intensity readings from a specimen, such as a cell, using three filter sets. The specific index of FRET is termed “the FRET ratio,” or FR. The 3³-FRET method also can be used to determine one or more of the following: the fraction of acceptor-tagged molecules that are bound by donor-tagged molecules; the relative affinity of that binding reaction; and the strength of FRET interaction when all acceptor-tagged molecules are bound by donor-tagged molecules. The latter determination enables estimates of the physical distance and/or orientation between interacting acceptor and donor fluorophore molecules. The method can be applied to determinations of FR, but may also be applied to many other quantitative FRET indices.

The method can be used to monitor inter- or intra-molecular interactions, detect analytes, identify polypeptide-binding partners from a library of expressed sequences (e.g., such as a cDNA library) and probe compounds for their ability to inhibit or enhance polypeptide binding. In a preferred aspect, the method is incorporated into an HTS assay for parallel screening of molelecular interactions across many samples.

The techniques and procedures described herein are performed according to conventional methods in the art and various general references which are provided throughout this document, including, but not limited to: Sambrook, et al., 1989, Molecular Cloning: A Laboratory Manual, 2d ed. (1989) Cold Spring Harbor Laboratory Press, Cold Spring Harbor, N.Y.; and Lakowicz, 1983, Principles of Fluorescence Spectroscopy, New York: Plenum Press.

Definitions

The following definitions are provided for specific terms which are used in the following written description.

As defined herein, a “polypeptide” refers to a polymer in which the monomers are amino acid residues which are joined together through amide bonds. When the amino acids are alpha-amino acids, either the L-optical isomer or the D-optical isomer can be used; however, the term also includes the polymers comprising unnatural amino acids such as beta-alanine, phenylglycine, and homo-arginine. For a general review, see, for example, Spatola, A. F., in Chemistry and Biochemistry of Amino Acids, Peptides and Proteins, B. Weinstein, ed., Marcel Dekker, New York, p. 267 (1983).

As used herein, “a fluorescent protein” refers to any protein capable of emitting light when excited with appropriate electromagnetic radiation. Fluorescent proteins include proteins having amino acid sequences that are either natural or engineered, such as the fluorescent proteins derived from Aequorea-related fluorescent proteins.

As used herein, “GFP” is the green fluorescent protein from the jellyfish, Aequorea victoria. As used herein, “CFP” and “ECFP” refer to the enhanced cyan fluorescent protein, a mutant variant of GFP, as described by Miyawaki, et al. (1997, Nature 388: 882-887). Likewise, as used herein, “YFP” and “EYFP” refer to the GFP variant enhanced yellow fluorescent protein, as described by Miyawaki, et al. (supra).

As used herein, a “nucleic acid” refers to DNA, RNA, DNA:RNA hybrids, single stranded or double stranded forms thereof, and includes modified or variant forms thereof.

As used herein, a “heterologous” region of a DNA construct is an identifiable segment of DNA within a larger DNA molecule that is not found in association with the larger molecule in nature. Thus, when the heterologous region encodes a mammalian gene, the gene will usually be flanked by DNA that does not flank the mammalian genomic DNA in the genome of the source organism (e.g., such as viral promoter sequences).

As used herein, a “donor molecule” refers to a fluorophore which when in the excited state can transfer energy to an acceptor molecule, provided that the donor fluorescence emission spectrum overlaps significantly with the acceptor absorption spectrum. An “acceptor molecule” refers to a fluorophore which, upon receiving energy from a donor molecule, can enter the excited state and emit a photon. A “suitable donor:acceptor pair” refers to a pairing of donor and acceptor fluorophores that satisfies the definitions of donor molecule and acceptor molecule.

As used herein, a “FRET signal” refers to the emission produced when an acceptor molecule receives energy from a donor molecule. Generally, energy transfer can only occur when two conditions are met: the donor and acceptor are separated by less than approximately 100 Å; and, the donor emission transition dipole and acceptor absorption transition dipole are not perpendicular (i.e., the orientation factor, κ², does not equal zero). A donor and acceptor molecule in “close proximity” refer to donor and acceptor molecules in sufficient proximity and at appropriate orientations to cause a FRET signal.

As used herein, a “light path” refers the geometrical distance between a light source and a light detector or photodetector.

As defined herein, a “ratio of acceptor and donor molar extinction coefficients scaled for the third (FRET) filter” refers to the ratio of acceptor and donor molar extinction coefficients at the preferred wavelength of the filter between the light source and specimen in the FRET filter set.

As used herein, a molecule which is “linked” to another molecule refers to a molecule which is stably coupled to another molecule, for example, by a covalent linkage. A “linked molecule” can be chemically conjugated to another molecule using methods routine in the art, or, if a polypeptide, can be engineered so as to be fused in frame with the other molecule (e.g., the covalent linkage may be an amide bond).

As used herein, a “modulator” of a molecular interaction refers to a compound which produces a statistically significant change in the interaction relative to the interaction as measured in the absence of compound.

As used herein, “a molecular interaction” refers to an intermolecular or an intramolecular interaction.

Advantages and Context of the 3³-FRET Process

FRET coupling between donor and acceptor fluorophores provides one of the most promising approaches for detecting polypeptide interactions in living samples, such as single cells. Donor and acceptor fluorophores can be chemically attached to two polypeptides (or, to different parts of the same polypeptide) within the sample, and FRET between the donor and acceptor then becomes an optical means of detecting whether the “tagged” polypeptides associate (i.e., are within close proximity). Detection and quantification of FRET signals generally relies on measurements of light in the visible or near-visible wavelengths, which is inherently non-invasive (i.e., does not require destruction of the sample). Thus, FRET can monitor polypeptide interactions in the setting of ultimate biological relevance—the living cell.

As a more specific illustration, consider the fluorescent proteins enhanced cyan fluorescent protein (ECFP) and enhanced yellow fluorescent protein (EYFP). Because ECFP and EYFP are small molecules (˜238 amino acids), they can easily be fused onto polypeptides of interest by standard techniques in recombinant engineering, and the resultant fusion proteins can be expressed in living cells. When ˜440 nm light illuminates molecules tagged with ECFP and/or EYFP, ECFP is preferentially excited, resulting in predominantly cyan fluorescence (˜480 nm) from the ECFP molecule. However, if ECFP and EYFP are held together at a distance of less than about 100, energetically excited ECFP can return to its ground state by transferring its energy to EYFP (i.e., via FRET) without emitting a fluorescent photon. An excited EYFP molecule can then relax and emit a yellow photon (˜535 nm), a phenomenon called sensitized EYFP emission (see, e.g., as described in Clegg, 1992, Methods Enzymol. 211: 353-358).

A shift from cyan to yellow fluorescence in a sample comprising a mixture of ECFP and EYFP thus indicates that ECFP and EYFP are within about 100 of each other. On the scale of typical proteins, separations of less than 100 generally imply that the two fluorophores, and by inference the polypeptides to which they are fused, are closely associated with one another. A sample, such as a cell, comprising molecules tagged with ECFP and EYFP, can thus be evaluated using an appropriate optical system to determine whether intermolecular interactions are taking place and/or whether compounds added to the sample are capable of modifying such interactions.

The detection of light required for quantification of donor:acceptor interactions requires an appropriate optical system, and many optical systems comprise filter sets. The individual filters which comprise a filter set transmit and/or reflect specific wavelengths of light. In the case of epifluorescent microscopes, these filter sets are usually combined as filter cubes. A wide spectrum of filter sets and/or cubes is available from most major manufacturers. Filter sets comprise one or more of the following: excitation filter, emission filter, and dichroic mirror (or, dichroic beamsplitter). Excitation filters permit only selected wavelengths from a light source to pass to a specimen, such as a cell. Emission filters are filters that block or absorb the excitation wavelengths and permit only selected emission wavelengths to pass to a photodetector, such as the eye, photomultiplier tube, or CCD camera. Emission filters generally suppress shorter wavelengths and have high transmission for longer wavelengths. Dichromatic mirrors are filters designed to reflect excitation wavelengths and transmit emission wavelengths. They are used in reflected light fluorescence illuminators and are positioned in the light path after the exciter filter but before the emission filter and are generally at a 45° angle with respect to light passing through the excitation filter and light passing through the emission filter. A filter set generally combines these elements to provide appropriate wavelengths of light to enable detection of a fluorophore. 3³-FRET processes the signals obtained from a combination of filter sets (or filter cubes) to produce an index of the strength of energy transfer between donor and acceptor molecules.

In practice, multiple challenges often complicate quantitation of the strength of FRET in living cells:

(1) Variable expression levels of fluorophore-tagged polypeptides: This makes it difficult to determine whether changes in fluorescence emission intensities are due to FRET or simply changes in fluorophore numbers.

(2) Incomplete fluorophore labeling of polypeptides, often arising from the expression of untagged, non-recombinant polypeptides by the cells.

(3) Inability to selectively excite donor fluorophores: This makes it difficult to prevent direct excitation of acceptor fluorophores, thus acceptor fluorescence emission due to direct excitation must be dissected from acceptor emission due to FRET.

(3) Similarly, the inability to selectively detect acceptor emission: To determine FRET from measurements of sensitized acceptor emission, fluorescence emission from the acceptor must be dissected from contaminating donor emission (i.e., donor “crosstalk”).

(4) Fluorescence emission from the acceptor must be dissected from contaminating donor emission (i.e., donor “crosstalk”).

(5) Some FRET assays require photo-destruction (e.g., photobleaching over many minutes) of the donor or acceptor fluorophores, which precludes measurements of FRET at different time points from the same sample.

These challenges apply to many different donor:acceptor pairs, as well as to the specific case of ECFP and EYFP as donor and acceptor, respectively. In particular, transient transfection with cDNA encoding ECFP- and EYFP-tagged polypeptides generally results in highly variable expression of fusion proteins. Even cells that have been manipulated to stably express CFP- and YFP-tagged polypeptides can demonstrate variable expression. Furthermore, wavelengths as low as 400 nm will persist in exciting EYFP directly, albeit less efficiently than for ECFP. Also, the broad emission spectrum of ECFP indicates that ECFP fluorescence emission will contribute yellow and green photons which must be distinguished from the yellow and green photons emitted by EYFP. In sum, existing tools within the art do not address all of these challenges, thus detection of FRET in living cells can be difficult, destructive of the sample, and/or time-consuming.

An additional, important challenge for FRET assays of polypeptide interactions is the inability to quantitate fractional binding of donor- and acceptor-tagged polypeptides. Specifically, different FRET signal strengths among samples could result from: different polypeptide binding affinities; or different donor:acceptor orientation/distance when the polypeptides are bound together; or a mixture of both. Thus, variations in the strength of the FRET signal could result from very different underlying causes that are difficult to distinguish from one another using standard tools in the art. For example, the multi-filter method, termed FRETN (Gordon, et al., 1998, Biophys J. 74:2702-2713), does not correct for variable expression levels of donor- and acceptor-tagged molecules. While the donor dequenching method (Miyawaki, and Tsien, 2000, Methods Enzymol. 327: 472-500) can account for variable expression levels, this method is nonetheless destructive. Moreover, FRETN, donor dequenching, and more general spectral dissection methods (e.g., Clegg, 1992, supra) do not provide means to quantitate fractional binding.

The 3³-FRET method provides a fast, simple, and nondestructive method for detecting and quantifying FRET, despite the challenges described above. One advantage of the 3³-FRET method is that it provides a way to nondestructively produce a quantitative index of the strength of FRET signal. The specific index of FRET is termed “the FRET ratio,” or FR. A second advantage of the 3³-FRET method is that it provides a way to nondestructively determine: the fraction of acceptor-tagged molecules that are bound by donor-tagged molecules; the relative affinity of a binding reaction; and the strength of FRET interactions when all acceptor-tagged molecules are bound by donor-tagged molecules. The latter determination enables estimates of the physical distance and/or orientation between interacting acceptor and donor fluorophore molecules to be obtained. This second advantage may be conveniently applied to determinations of FR, but may also be applied to many other quantitative FRET indices.

The 3³-FRET Process

To overcome the challenges for quantifying FRET, as enumerated in the section above, one must be able to decompose the individual signals that contribute to the detector output when the FRET filter set is engaged. To be concrete, the requisite decomposition for the ECFP/EYFP FRET pair is described, although the necessary capabilities generalize to numerous FRET pairs.

First, one must be able to distinguish that portion of the detector output due to ECFP fluorescence in the yellow color range detected by the FRET filter set. Second, of the remaining signal due to EYFP emission, one must be able to distinguish that portion due to direct excitation versus that portion due to FRET. 3³-FRET accomplishes these objective while fully exploiting simplifications made possible by the particular spectral properties of many FRET pairs, including ECFP and EYFP. The suitability of the 3³-FRET method for the ECFP/EYFP pair is especially advantageous, given that this is the leading genetically-encoded FRET pair currently available. For example, the EBFP/EGFP FRET pair is not as favorable due to the relatively poor quantum yield of EBFP (Miyawaki et al., 1997, supra). FRET pairs involving red-shifted fluorescent proteins, such as DsRed, often suffer from slow fluorophore maturation and intracellular aggregation (see Lauf, et al., 2001, FEBS Letters 498:11-15). Having underscored the comparative advantages of the ECFP/EYFP FRET pair at the current time, it is also important to emphasize that the 3³-FRET method generalizes to many other suitable FRET pairs. Hence, when other, more favorable genetically-encoded FRET pairs become available, the 3³-FRET method will likely be of considerable advantage for quantifying FRET from these pairs.

The invention provides a method for detecting a FRET signal from a specimen containing suitable donor-tagged and acceptor-tagged molecules utilizing 3³-FRET. 3³-FRET involves “optical dissection” by obtaining sequential intensity readings from a single specimen (e.g., such as a cell) at a time, using measurements made with the three filter sets. Simple equations manipulate readings from each of the filter sets to specify a unitless index of FRET called the FRET ratio (FR). FR bears a linear relation to FRET efficiency E, described further below.

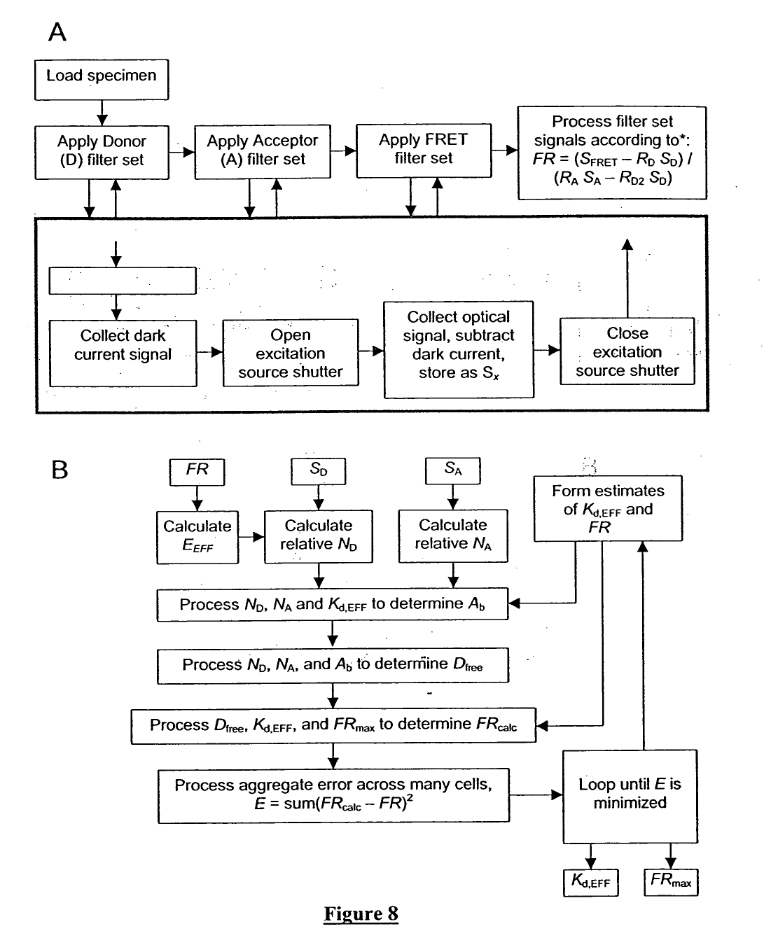

Preferably, sequential light intensity readings are obtained from the specimen using an optical system that can sequentially engage three filter sets or cubes (FIG. 8A). One filter set preferentially detects donor emission, one filter set preferentially detects acceptor emission, and one filter set detects emissions from both donor and acceptor fluorophores.

An exemplary optical system for use in the method comprises a light source for providing excitation light to the specimen; a detector; a specimen holder for positioning the specimen in a suitable position to receive light from the light source and to transmit light emitted by the specimen to the detector; and a filter set holder for sequentially receiving first, second, and third filter sets and for positioning each of the filters. Preferably, the optical system is selected from the group consisting of an epifluorescence microscope, a confocal microscope, a flow cytometer, and a plate reader.

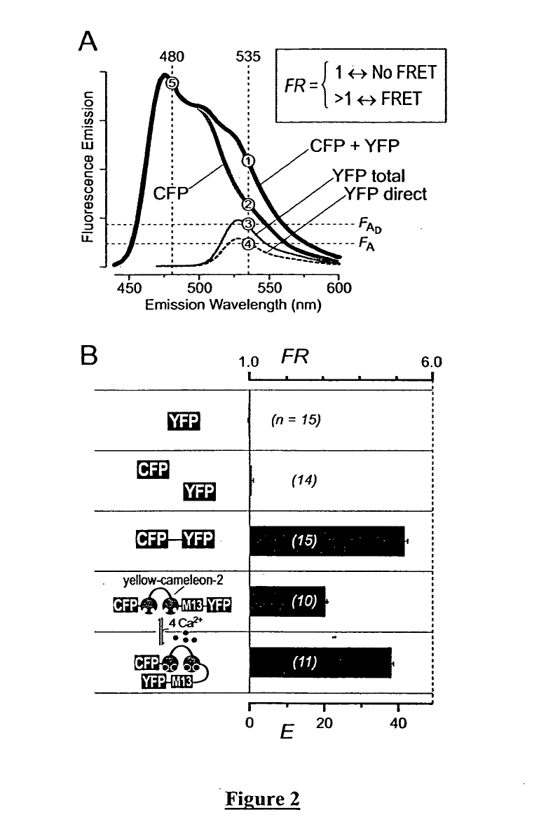

Having reviewed the overall physical setup pertaining to the 3³-FRET method, the essential qualitative principle of the process is described below in the context of an ECFP/EYFP (however, as discussed above, the method can be generally applied to an suitable donor:acceptor FRET pairs). FIG. 2A shows a fluorescence emission spectrum produced by illuminating a cell expressing both ECFP and EYFP with light at 440 nm. The double-humped shape results from superposition of individual ECFP (thick line) and EYFP (thin line) spectra. FRET alters this spectrum by decreasing the ECFP (energy donor) peak near 480 nm and enhancing the EYFP (energy acceptor) peak near 535 nm. FRET can therefore be nondestructively dissected from the enhanced EYFP emission at 535 nm by eliminating signal from secondary EYFP emissions due to direct excitation (dashed line) from total EYFP emission (thin line) due to both FRET and direct excitation.

Emission at 535 nm (FIG. 2A, number 1) is the sum of CFP emission (number 2) and YFP emission (number 3), a portion of which is due to direct excitation (number 4). To dissect these components, 3³-FRET employs filter sets that isolate CFP and YFP signals from a cell expressing both fluorophores. The CFP filter set excites both fluorophores but measures fluorescence where only CFP emits (number 5). Multiplying this measurement by a predetermined constant provides CFP emission at 535 nm (number 2), which is subtracted from number (1) to determine total YFP emission (F_{A}_{_{D}}; number 3). Similarly, the YFP filter set measures near exclusive YFP emission by preferential excitation of YFP. Multiplying this measurement by a constant gives YFP emission due to direct excitation (F_{A}; number 4). Finally, the FRET ratio (FR=F_{A}_{_{D}}/F_{A}) is produced, a unitless index equal to the fractional increase in YFP emission due to FRET. As the amount of FRET increases, FR rises above unity, reaching a theoretical maximum of ˜12 for a ECFP/EYFP pair exhibiting 100% FRET efficiency (E).

Quantitative Representation of Optical System Properties and Fluorescence

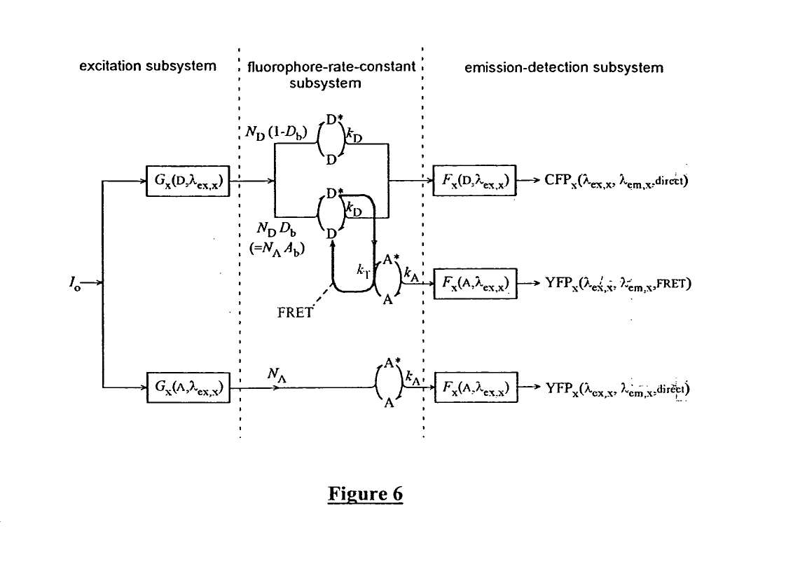

To aid in detailed understanding of the algorithms that process multiple filter set measurements in order to produce FR, it is convenient to model the properties of an optical detection system (e.g., such as an epifluorescence microscope) and fluorophores by the following formalism. In a FRET system, there are two types of fluorophores, donor (D) (e.g., ECFP) and acceptor (A) (e.g., EYFP), each of which can exist in a ground state (D, A) or in excited states (D*, A*), as shown in FIG. 6. In a field of view of the detection system, there are N_{A} and N_{D} donor and acceptor molecules, respectively. D_{b} represents the fraction of donor molecules bound by an acceptor, and A_{b} is the fraction of acceptor molecules bound by a donor. It is assumed that no FRET occurs between unassociated donor and acceptor molecules.

The excitation subsystem models the effects of properties of components of an optical detection system used to perform FRET measurements on the excitation rate of a fluorophore. In particular, the subsystem accounts for the effects of properties of an excitation light source, excitation filter, and dichroic mirror (e.g., such as are found in an epifluorescence microscope) on excitation rates.

The excitation rate (in units of transitions per second) of a single ground-state fluorophore may be represented by I₀Gₓ(y,λ_{ex,x}), where I₀ is the overall intensity of the xenon lamp (over all wavelengths), x specifies which of three filter sets is being used (D, A, or FRET), y specifies a donor or acceptor molecule (D or A) is being evaluated, and λ_{ex,x} is the predominant wavelength of excitation light (determined mainly by the excitation filter of filter set or cube x). Gₓ(y,λ_{ex,x}) is thus a constant that incorporates spectral properties of a light source used in an optical detector, such as a epifluorescence microscope, optical properties of the excitation filter and dichroic mirror of filter set or cube x, and wavelength-dependent absorption properties of the fluorophore in question as given by a molar extinction coefficient (ε_{y}(λ)).

The fluorophore-rate-constant subsystem models behavior of fluorophores as a state diagram with interstate transitions governed by various rate constants. The rate constants relating to emission of fluorescent photons (k_{D} and k_{A}) are functions of intrinsic properties of donor and acceptor molecules, and are independent of the wavelengths used for excitation or detection of emission. The rate constant pertaining to resonance energy transfer (k_{T}) is a function of many factors, including distance and orientation between bound donor and acceptor molecules, as well as overlap between emission and excitation spectra of donor and acceptor molecules, respectively. However, k_{T} is also independent of the wavelengths used for excitation or detection of emission.

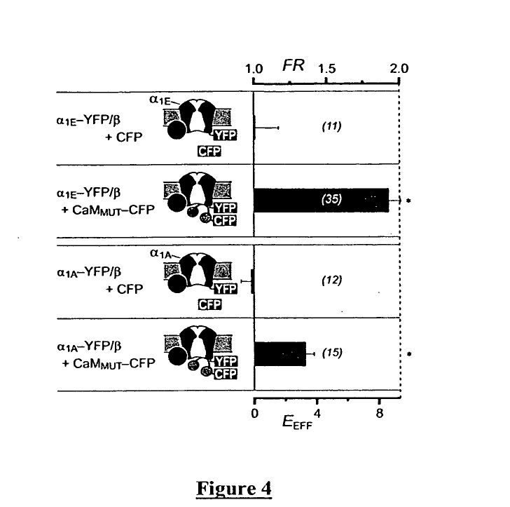

The rate-constant model, shown in FIG. 4, describes the probabilities of occupying A, A*, D, and D* states, and the probability flux of transitions among the various states. For purposes of calculating fluorescence emission, only the steady-state behavior of the system is considered because measurement times are far larger than the characteristic relaxation times. A standard assumption for derivation of FRET equations is that the system is in the “low-excitation limit,” where excitation power is low enough that the steady-state probabilities of being in D or A (P_{D} or P_{A}) are essentially unity and this has been experimentally verified for this system. The steady-state probability of occupying D* is:

P_{D}*=(1−D_{b})·Iₒ·Gₓ(D,λ_{ex,x})/k_{D}+D_{b}·Iₒ·Gₓ(D,λ_{ex,x})/k_{T}+k_{D}) [A1]

where k_{T} is the rate constant for FRET between donor and acceptor molecules (all rate constants in units of s⁻¹), and k_{D} is the rate constant for the emission of non-FRET relaxation from D* to D. The rate constants k_{T} and k_{D} are independent of wavelength, but k_{T} is a function of donor-acceptor distance according to the Förster equation (Förster, 1948, Ann. Physik. 2: 55; Förster, 1960, Rad. Res. Suppl. 2: 326). Likewise, under the low-excitation limit, the steady-state probability of occupying A* is given by

P_{A}*=Iₒ·Gₓ(A,λ_{ex,x})/k_{A}+A_{b}·[Iₒ·Gₓ(D,λ_{ex,x})/(k_{T}+K_{D})]·k_{T}/k_{A} [A2]

where k_{A} is the wavelength-independent rate constant for (non-FRET) relaxation from A* to A. The first term (Gₓ(A,λ_{ex,x})) is a measure of direct excitation of the acceptor by the light source of an optical system (e.g., such as a xenon lamp), which occurs regardless of whether a donor is bound, while the second term (Gₓ(A,λ_{ex,x})) is a measure of FRET excitation of the acceptor (which only can occur if donor is bound).

The emission-detection subsystem accounts for properties of the emission filter, dichroic mirror, photomultiplier electronics, as well as fluorophore emission spectrum and quantum yield. The three output signals on the right of FIG. 6 are those that comprise aggregate fluorescence output obtained with any of the filter sets or cubes.

In one aspect, the rate of excited donor relaxations which can possibly give rise to fluorescence emissions by acceptor molecules is represented as k_{D} P_{D}*. The fluorescence output from the photodetector of an optical system (e.g., such as a photomultiplier tube or PMT), in mV output per second, arising from excited donor relaxations is represented as:

N_{D}·k_{D}·P_{D}*·Fₓ(D,λ_{em,x})

where N_{D} is the number of donor molecules in the field of view, λ_{ex,x} the predominant wavelength or wavelength range of the output segment of filter set or cube x, and Fₓ(D,λ_{em,x}) is an “output transfer function,” corresponding to the signal actually produced by the optical detector, with units of mV per non-FRET donor relaxation. Fₓ(D,λ_{em,x}) is a constant that incorporates the emission spectrum and quantum yield of the donor, the dichroic mirror and emission filter optical properties of filter set or cube x, and frequency-dependent sensitivity of the photodetector.

In order to make the following development of the algorithms underlying 3³-FRET more concrete, the specific case of ECFP as donor and EYFP as acceptor is described. These same algorithms can be generalized for any suitable donor:acceptor FRET pair.

Thus, inserting Equation A1 into the expression above yields the full equation, A3, for fluorescence output resulting from donor fluorescence, as measured with a filter set or cube x:

CFPₓ(λ_{ex,x}, λ_{em,x},direct)=N_{D}·k_{D}·[((1−D_{b})/k_{D})+(D_{b}/(k_{T}+k_{D}))]·Iₒ·Gₓ(D,λ_{ex,x})·Fₓ(D,λ_{em,x} [A3]

Likewise, analogous reasoning and Equation A2 provide the full equation for fluorescence output resulting from acceptor fluorescence, as measured with filter set or cube x. This entity is given by two terms relating to direct and FRET excitation (A4 and A5, respectively).

YFPₓ(λ_{ex,x},λ_{em,x},direct)=N_{A}·k_{A}·[IₒGₓ(A,λ_{ex,x})/k_{A}]·Fₓ(A,λ_{em,x}) [A4]

YFPₓ(λ_{ex,x},λ_{em,x},FRET)=N_{A}·A_{b}·[IₒGₓ(D,λ_{ex,x})/k_{T}+k_{D})]·k_{T}·Fₓ(A,λ_{em,x}) [A5]

The actual fluorescence signal output obtained from a given sample using a particular optical filter set or cube can be denoted by the descriptor Sₓ (specimen), where x is the name of the filter set or cube (e.g., ECFP, EYFP, FRET) and the specimen is either the donor (D), acceptor (A), or both (DA). Alternatively, as a conceptual aid in the detailed derivations below, the longer specifier of the fluorescence signal output Sₓ (specimen, λ_{ex,x}, λ_{em,x}) (which is equivalent to Sₓ (specimen)) can be used. In the longer specifier, excitation wavelength is denoted herein as λ_{ex,x}, while the emission wavelength is denoted as λ_{em,x} as detected by a photo detection device (e.g., a CCD camera). Thus, the signal output obtained from ECFP with the ECFP filter set or cube is S_{CFP} (D, 440,480). The added information in the longer specifier serves as a reminder of dominant operating features of the filter set employed.

Measurement Ratios for Transformation of Optically Isolated Signals from Donor or Acceptor

Certain fluorescence measurements obtained from a mixture of both donor and acceptor molecules can be attributed primarily to donor or acceptor only. To transform such “optically isolated” fluorescence signals into those that would be in effect using excitation and emission wavelengths where both donor and acceptor fluorescence would be appreciable (such as near 535 nm where sensitized acceptor fluorescence of EYFP occurs), three predetermined ratios of measurements for acceptor only, or donor only.

$$\begin{matrix}{R}_{A1} = \frac{{S}_{FRET} ( A , 440 , 535 )}{{S}_{YFP} ( A , 500 , 530 LP )} = \frac{{G}_{FRET} ( A , 440 ) \cdot {F}_{FRET} ( A , 535 )}{{G}_{YFP} ( A , 500 ) \cdot {F}_{YFP} ( A , 530 LP )} & [ A6 ] \\ {R}_{D1} = \frac{{S}_{FRET} ( D , 440 , 535 )}{{S}_{CFP} ( D , 440 , 480 )} = \frac{{G}_{FRET} ( D , 440 ) \cdot {F}_{FRET} ( D , 535 )}{{G}_{CFP} ( D , 440 ) \cdot {F}_{CFP} ( D , 480 )} & [ A7 ] \\ {R}_{D2} = \frac{{S}_{YFP} ( D , 500 , 530 LP )}{{S}_{CFP} ( D , 440 , 480 )} = \frac{{G}_{YFP} ( D , 500 ) \cdot {F}_{YFP} ( D , 530 LP )}{{G}_{CFP} ( D , 440 ) \cdot {F}_{CFP} ( D , 480 )} & [ A8 ]\end{matrix}$$

Note that these ratios are independent of the excitation intensity I₀ and the number of donor or acceptor molecules in the field of view N_{D} or N_{A}. Thus, they can be determined in cells in which acceptor or donor alone are expressed, and then applied to calculations of equations representing the signals obtained from cells in which mixtures of acceptor and donor are expressed.

The utility of these ratios can be illustrated by a simple case example. Suppose a ECFP filter set or cube is used to obtain a fluorescence measurement from a cell expressing both a donor and acceptor. Because EYFP does not detectably emit photons in the 480 nm range, the ECFP filter set or cube measurement S_{CFP}(DA,440,480) is equivalent to the contribution due to ECFP fluorescence alone, or CFP_{CFP}(440,480,direct), as calculated from Equation A3. S_{CFP}(DA,440,480) can be related to CFP_{FRET}(440,535,direct), or the ECFP fluorescence that would be present using a FRET filter set or cube, as follows:

CFP_{FRET}(440,535,direct)=R_{D1}·S_{CFP}(DA,440,480) [A9]

Thus, determining the ratio R_{D1} provides a way to transform the optically isolated ECFP signal, S_{CFP}(DA,440,480), into the ECFP contribution to fluorescence at 535 nm, where both EYFP and ECFP fluorescence are appreciable.

A remarkable feature of the transformation is that it is rather exact, regardless the concentrations of donor and acceptor in the field of view, the possibility of binding and FRET between donor and acceptor molecules, the excitation power, and the inevitable idiosyncrasies of the optical filter set or cubes involved. These factors have all been incorporated into Equations A3 and A7, which were used to solve for Equation A9. This feature of exactness and generality pertains to all subsequent calculations as well.

Complete Determination of FRET Ratio (FR) by the 3³-FRET Method

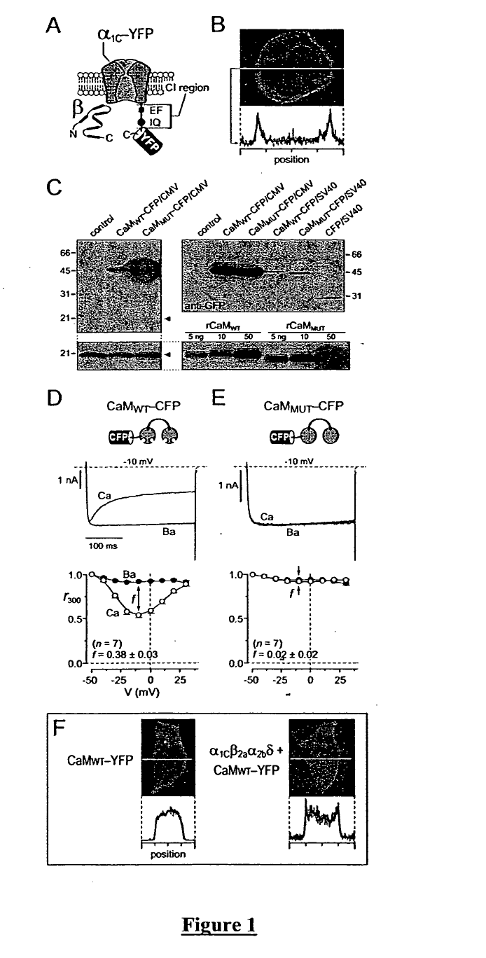

In one aspect, the FRET ratio (FR) to be produced by the 3³-FRET method, is defined as

$$FR ≡ \frac{{YFP}_{FRET} ( 440 , 535 , FRET ) + {YFP}_{FRET} ( 440 , 535 , direct )}{{YFP}_{FRET} ( 440 , 535 , direct )} \qquad [ A10 ]$$

where the terms refer to EYFP fluorescence due to direct and FRET excitation, as defined in Equations A4 and A5. The numerator of the expression is easily determined from the experimental measurement S_{FRET}(DA,440,535) by considering its constituent components (see, e.g., as shown in FIG. 1A) and Equations A3-A5:

S_{FRET}(DA,440,535)=YFP_{FRET}(440,535,FRET)+YFP_{FRET}(440,535,direct)+CFP_{FRET}(440,535,direct) [A11]

Solving the equation, using the measure of the third term, CFP_{FRET}(440,535,direct), as determined from Equation A9, the numerator of the FR expression in Equation A 10 is experimentally determined as

YFP_{FRET}(440,535, FRET)+YFP_{FRET}(440,535, direct)=S_{FRET}(DA,440,535)−R_{D1}·S_{CFP}(DA,440,480) [A12]

To solve for YFP_{FRET}(440,535,direct), the denominator of the FR expression in Equation A10, the EYFP filter set or cube measurement S_{YFP}(DA,500,530LP) can be expressed in terms of its three constituent components (see, FIG. A1). With reference to Equations A3-A5, an expression strictly analogous to Equation A11 can be represented as follows:

S_{YFP}(DA,500,530LP)=YFP_{YFP}(500,530LP, FRET)+YFP_{YFP}(500,530LP, direct)+CFP_{YFP}(500,530LP,direct) [A13]

The second term dominates the expression, consistent with the near selective excitation of EYFP with the EYFP filter set or cube. The third term is considerably smaller, while the first term is even smaller, and negligible from a practical standpoint. In practice, the first term can be ignored.

Just as with Equation A9, Equations A3 and A8 can be combined to specify the third term as a function of experimentally determined measures, according to:

CFP_{YFP}(500,530LP,direct)=R_{D2}·S_{CFP}(DA,440,480) [A14]

Solving Equation A13 for YFP_{YFP}(500,530LP,direct) and substituting from Equation A14 yields:

YFP_{YFP}(500,530LP,direct)=S_{YFP}(DA,500,530LP)−R_{D2}·S_{CFP}(DA,440,480)−YFP_{YFP}(500,530LP, FRET) [A15]

With the aid of Equations A4 and A6, the product R_{A1}YFP_{YFP} (500,530LP,direct) can be shown to be exactly equal to YFP_{FRET}(440,535,direct). Hence, multiplying Equation A15 by R_{A1}, yields

YFP_{FRET}(440,535,direct)=R_{A1}·[S_{YFP}(DA,500,530LP)−R_{D2}·S_{CFP}(DA,440,480)]−R_{A1}·YFP_{YFP}(500,530LP,FRET) [A16]

Equation A5 allows us to relate YFP_{YFP}(500,530LP,FRET) to YFP_{FRET}(440,535,FRET) by the relation

$$\begin{matrix}{R}_{A1} \cdot {YFP}_{YFP} ( 500 , 530 LP , FRET ) = \\ {[ \frac{{G}_{YFP} ( D , 500 )}{{G}_{FRET} ( D , 440 )} \cdot \frac{{G}_{FRET} ( A , 440 )}{{G}_{YFP} ( A , 500 )} ]}_{{1 \; 4 \; 4 \; 4 \; 4 \; 4 \; 2 \; 4 \; 4 \; 4 \; 4 \; 4 \; 3 }_{Y}} \cdot {YFP}_{FRET} ( 440 , 535 , FRET )\end{matrix} \qquad [ A17 ]$$

Substituting Equation A 17 into Equation A 16 yields

YFP_{FRET}(440,535,direct)=R_{A1}·[S_{YFP}(DA,500,530LP)−R_{D2}·S_{CFP}(DA,440,480)]−Y·YFP_{FRET}(440,535,FRET) [A18]

Finally, Equations A12 and A18 can be solved simultaneously to give the denominator term for FR (in Equation A10), in terms of experimentally measurable entities, as given by

$$\begin{matrix}{YFP}_{FRET} ( 440 , 535 , direct ) = \frac{1}{( 1 - Y )} \cdot {R}_{A1} \cdot \\ [ {S}_{YFP} ( DA , 500 , 530 LP ) - \\ {R}_{D2} \cdot {S}_{CFP} ( DA , 440 , 480 ) ] - \\ \frac{Y}{( 1 - Y )} \cdot [ {S}_{FRET} ( DA , 440 , 535 ) - \\ {R}_{D1} \cdot {S}_{CFP} ( DA , 440 , 480 ) ]\end{matrix} \qquad [ A19 ]$$

Substituting Equations A12 and A19 into the FR expression in Equation A10 provides the FRET ratio expressed in terms of 3³-FRET experimental measures. The complete relation is:

$$FR = \frac{\begin{matrix}[ 1 - Y ] \cdot [ {S}_{FRET} ( DA , 440 , 535 ) - \\ {R}_{D1} \cdot {S}_{CFP} ( DA , 440 , 480 ) ]\end{matrix}}{\begin{matrix}{R}_{A1} \cdot [ {S}_{YFP} ( DA , 500 , 530 LP ) - {R}_{D2} \cdot {S}_{CFP} ( DA , 440 , 480 ) ] - \\ Y \cdot {YFP}_{FRET} ( 440 , 535 , FRET )\end{matrix}} \qquad [ A20 ]$$

The magnitude of Y turns out to be exceedingly small, and can be estimated from the ratio of molar extinction coefficients for ECFP and EYFP (ε_{CFP}(λ) or ε_{YFP}(λ)), as given by

$$Y = \frac{{G}_{CFP} ( D , 500 )}{{G}_{CFP} ( D , 440 )} \cdot \frac{{G}_{YFP} ( A , 440 )}{{G}_{YFP} ( A , 500 )} \approx [ \frac{{ϵ}_{CFP} ( 500 )}{{ϵ}_{CFP} ( 440 )} ] \cdot [ \frac{{ϵ}_{YFP} ( 440 )}{{ϵ}_{YFP} ( 500 )} ] \qquad [ A21 ]$$

The ratios of molar extinction were determined for the terms in brackets, using excitation spectra for ECFP and EYFP. These ratios indicate that Y<0.001. All FRs in FIGS. 2-5 were calculated for Equation A20 and by setting Y=0.001. The FR values processed in this manner were in all instances less than 0.1% different than FR values obtained when Y=0. Hence, for all practical purposes with a ECFP-EYFP pair, Y can be set to zero, yielding the FR Equation A22.

$$FR = \frac{[ {S}_{FRET} ( DA , 440 , 535 ) - {R}_{D1} \cdot {S}_{CFP} ( DA , 440 , 480 ) ]}{{R}_{A1} \cdot [ {S}_{YFP} ( DA , 500 , 530 LP ) - {R}_{D2} \cdot {S}_{CFP} ( DA , 440 , 480 ) ]} \qquad [ A22 ]$$

This 3³-FRET determination of FR holds true, regardless of the concentrations of donor and acceptor in the field of view, the possibility of binding and FRET between donor and acceptor molecules, the excitation power, and the inevitable idiosyncrasies of the optical filter sets or cubes involved. These factors have all been incorporated into all of the equations from which Equation A22 is derived, and they cancel out in the final analysis.

Having described the complete 33-FRET process, a very important expression parameter to optimize in using 3³-FRET becomes clear. Since FR relies on EYFP emission, EYFP must be attached to the presumed limiting moiety in a given interaction. Otherwise, the fraction of EYFP-tagged molecules with an ECFP-tagged molecule bound may be low, thus producing little FRET as gauged by the 3³-FRET process.

Simplified Nomenclature and Intuitive Synopsis of 3³-FRET Principles

Having delineated the complete process above, it is worth intuitively revisiting the essential principles of 3³-FRET, now substituting back the more compact nomenclature for fluorescence output, and use Sₓ (specimen) in place of Sₓ (specimen, λ_{ex,x,}λ_{em,x}). Again, ECFP and EYFP are used as specific examples of methods that generalize to many donor:acceptor pairs. Recall that, in one aspect, a measurement of light received by a photodetector of an optical system from a cube or filter set can be compactly represented as S_{CUBE} (SPECIMEN), where CUBE denotes a particular filter set or cube (ECFP, EYFP or FRET) and SPECIMEN indicates the nature of a sample being evaluated using the filter set or cube, for example, a sample (e.g., a cell) expressing donor only (D; ECFP), acceptor only (A; EYFP) or both (DA, FRET). As shown in FIG. 2A (number 1), S_{FRET}(DA) is the sum of both ECFP emission (number 2) and EYFP emission (number 3), a portion of which is due to direct excitation (number 4). The key to dissecting these components requires obtaining measurements from both ECFP and EYFP filter sets or cubes (S_{CFP}(DA) and S_{YFP}(DA)), to optically isolate ECFP and EYFP signals received from a sample (e.g., a cell) expressing both fluorophores.

S_{CFP}(DA) (number 5) represents a filter set or cube which transmits light to a sample (e.g., such as a cell) which excites ECFP and EYFP but which transmits fluorescence to a detector only in the range that ECFP emits. The term can be multiplied by predetermined constant, R_{D1} to determine what the contribution of ECFP emission is at 535 nm (number 2). Subtracting this value from S_{FRET}(DA) leaves F_{A}_{_{D}}. Similarly, multiplying S_{YFP}(DA) which represents a filter set or cube which nearly exclusively excites EYFP but not ECFP, by a constant (R_{A1}), yields the component of S_{FRET}(DA) due to direct excitation of EYFP, or F_{A}. Constants R_{D1}, R_{D2} and R_{A1} are pre-determined from measurements applied to cells expressing only ECFP or EYFP. The ratio of F_{A}_{_{D}} to F_{A} provides the FRET ratio, FR which can be represented now in compact form as:

$$FR = \frac{{F}_{{A}_{D}}}{{F}_{A}} = \frac{[ {S}_{FRET} ( DA ) - {R}_{D1} \cdot {S}_{CFP} ( DA ) ]}{{R}_{A1} \cdot [ {S}_{YFP} ( DA ) - {R}_{D2} \cdot {S}_{CFP} ( DA ) ]}$$

Preferably, averages of light emissions obtained from specimens not containing donor or acceptor molecules also are obtained and subtracted from experimental values for each filter set or cube; all Sₓ(specimen) measurements are thus background subtracted. FR bears a linear relationship to FRET efficiency E and becomes greater than unity with increasing FRET. Specifically, FRET efficiency (E) is determined from FR by

E=(FR−1)[ε_{YFP}(440)/ε_{CFP}(440)]

where the bracketed term is the ratio of EYFP and ECFP molar extinction coefficients, scaled for the FRET filter set or cube excitation filter (Selvin, 1995, Methods Enzymol. 246: 300-334). This transformation can be derived from standard results in the field.

As FR can be directly transformed to efficiency E, FR can also be correlated with the physical distance between donor-acceptor pairs using the Forster equation (Selvin, 1995, supra), R=R₀(E⁻¹−1)^{1/6}, wherein the Förster distance R₀=49 (Patterson, et al., 2000, Anal Biochem. 284: 438-440). This transformation presumes that each acceptor-tagged molecule is bound by a donor-tagged molecule, and that donor-acceptor orientations are randomized. The binding assumption will be further expanded in a section below.

Experimental Verification of Sensitive, Single-Cell FRET Detection by 3³-FRET

Control experiments verify that 3³-FRET provides sensitive and selective detection of FRET (FIG. 2B). Averaged data from individual cells expressing only EYFP gave an FR˜1, as expected for this trivial case when no donor is present. Cells co-expressing ECFP and EYFP also showed no FRET, arguing against confounding concentration dependent artifacts such as dimerization or trivial reabsorption. A significant increase in FR was observed for cells expressing a ECFP-EYFP concatemer in which ECFP and EYFP are linked together by a short polypeptide and thereby held within 100 Å. Finally, the FRET-based calcium-sensor yellow cameleon-2 (Miyawaki, et al., 1997, Nature 388: 882-887) showed the expected Ca²⁺-dependent increase in FR. See Example 1 below for further discussion.

The relationship between FR and E, described above, enabled another specific validation test of the 3³-FRET method. E also can be determined by measuring dequenching of donor emission following near complete acceptor photodestruction (while sparing the donor) by several minutes of strong illumination through an excitation filter that excites the acceptor but not the donor (see, e.g., as described in Miyawaki, and Tsien, 2000, Methods Enzymol. 327: 472-500). This approach complements 3³-FRET but is slower, destructive, and entails unavoidable collateral photobleaching (e.g., of cells in addition to one being analyzed). By comparing this value of E with FR for single cells expressing ECFP-EYFP, ε_{YFP}(440)/ε_{CFP}(440) was experimentally found to be 0.096, which is within 3% of the predicted value based on published extinction coefficients for ECFP and EYFP (Patterson, et al., 2001, J Cell Sci 114: 837-838).

Characterizing Properties of Binding Between Donor- and Acceptor-Tagged Molecules Using 3³-FRET

As described above, the 3³-FRET process can be used to quantify FRET, and therefore the presence or absence of interaction between donor and acceptor molecules. The process can also be used to provide information about the properties of binding between donor and acceptor molecules, under the presumption that donor-acceptor interaction follows a 1:1 stoichiometry. The steps involved are summarized in FIG. 8B. A key underlying principle is that ECFP and EYFP filter-set or cube measurements, as processed according to the invention, provide a method for estimating the relative concentrations of ECFP- and EYFP-tagged molecules in single cells. When combined with estimation of a single Langmuir binding function, the fraction of EYFP-tagged molecules associated with ECFP-tagged partners can be calculated. The calculated fraction can be used to predict a FRET ratio (FR=F_{A}_{_{D}}/F_{A}) according to formula A23 described further below.

$$FR = 1 + {[ \frac{{G}_{FRET} ( D , 440 )}{{G}_{FRET} ( A , 440 )} \cdot E ]}_{{1 \; 4 \; 4 \; 4 \; 2 \; 4 \; 4 \; 43 }_{Δ {FR}_{max}}} \cdot {A}_{b} \qquad [ A23 ]$$

where E=k_{T}/(k_{T}+k_{D}) is defined as the FRET efficiency of a donor-acceptor pair, and the ratio of G_{FRET}(D,440)/G_{FRET}(A,440) is essentially equal to the ratio of molar extinction coefficients ε_{CFP}(440)/ε_{YFP}(440).

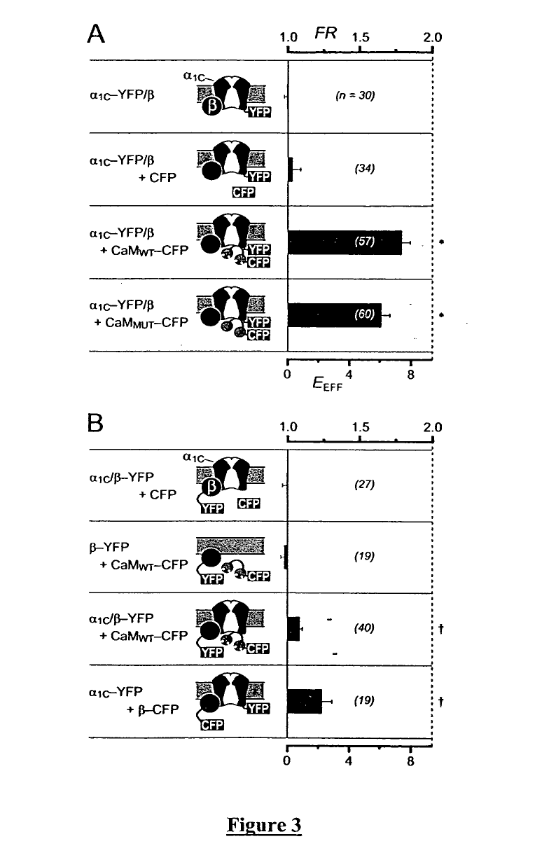

This equation can be used to address the scenario where low expression levels of donor and acceptor result in unpaired donor and/or acceptor molecules and highlights three important features of incomplete labeling of acceptor molecules. First, the measured FR varies linearly with an increasing fraction of acceptor bound to donor, according to slope ΔFRₘₐₓ. Second, the equation also indicates that the efficiencies calculated in FIG. 3 are actually “effective efficiencies” E_{eff}=E·A_{b}. Finally, to calculate the “true” efficiency E, ΔFRₘₐₓ must be estimated from some type of regression analysis based upon measured FR as a function of A_{b}. E would be required to constrain actual distances between donor and acceptor moieties according to the Förster equation. The last point underscores the need for an experimental estimate of A_{b}.

To estimate A_{b} from 3³-FRET measurements on a single cell, A_{b} can be represented by the classic binding equation

A_{b}=1/(1+2·K_{d}/[D_{free}]) [A24]

assuming acceptor molecules which are membrane associated (e.g., such as ion channels), free donor molecules which are soluble cytoplasmic moieties (like tagged CaM), and a stoichiometry of donor-acceptor binding of 1:1. In this equation, K_{d} is the dissociation constant (in M units), [D_{free}] is the concentration of free (unbound) donor molecules (in M units), and the factor of 2 relates to the fact that donor molecules can only bind to acceptor from the cytoplasmic side of the membrane. This can be restated in terms of the total number of donor and acceptor molecules in a cell (which is always within a field of view) as

A_{b}=1/[1+2·K_{d}·V·N_{avogadro}/(N_{D}−A_{b}·N_{A})] [A25]

where N_{avogadro} is Avogadro's number, N_{D} and N_{A} are number of donor and acceptor molecules in the cell, and V is the volume of the cell (in liters). Solving this equation for A_{b}, yields

$${A}_{b} = \frac{\begin{matrix}{N}_{D} + {N}_{A} + ( 2 \cdot {N}_{avogadro} \cdot {K}_{d} \cdot V ) - \\ \sqrt{{( {N}_{D} + {N}_{A} + ( 2 \cdot {N}_{avogadro} \cdot {K}_{d} \cdot V ) )}^{2} - 4 \cdot {N}_{D} \cdot {N}_{A}}\end{matrix}}{2 \cdot {N}_{A}} \qquad [ A26 ]$$

This provides an optical means of estimating N_{D} and N_{A}. From Eqs. A9 and A18, an optical means of calculating CFP_{FRET}(440,535,direct) and YFP_{FRET}(440,535,direct) is obtained. From Eqs. A3 and A4, these are related to N_{D} and N_{A} by the equations

CFP_{FRET}(440,535,direct)=N_{D}·k_{D}·[((1−D_{b})/k_{D})+(D_{b}/(k_{T}+k_{D}))]·Iₒ·G_{FRET}(D,440)·F_{FRET}(D,535) [A27]

YFP_{FRET}(440,535,direct)=N_{A}·Iₒ·G_{FRET}(A,440)·F_{FRET}(A,535) [A28]

From the definition of E_{eff} above, Equation A27 can be recast into the very useful form below:

CFP_{FRET}(440,535,direct)=[N_{D}−E_{eff}·N_{A}]·Iₒ·G_{FRET}(D,440)·F_{FRET}(D,535) [A29]

The G and F terms in Eqs. A28 and A29 can be estimated by

G_{FRET}(A,440)·F_{FRET}(A,535)≈C·[ε_{A}(λ)]_{λ=430-450 nm}·[ƒ_{A}(λ)]_{λ=505-575 nm} [A30]

G_{FRET}(D,440)·F_{FRET}(A,535)≈C·[ε_{D}(λ)]_{λ=430-450 nm}·[ƒ_{D}(λ)]_{λ=505-575 nm} [A31]

where C is a constant, [ε_{A}(λ)]_{λ=430-450 nm} is the average molar extinction coefficient of EYFP over the bandwidth of the FRET filter set or cube excitation filter (430-450 nm); [ε_{D}(λ)]_{λ=430-450 nm} is the average molar extinction coefficient of ECFP over the same bandwidth; [ƒ_{A}(λ)]_{λ=505-575 nm} is the average value of the EYFP emission spectrum over the bandwidth of the FRET filter set or cube emission filter (505-575 nm); and [ƒ_{D}(λ)]_{λ=505-575 nm} is the average value of the ECFP emission spectrum over the same emission filter bandwidth. Prior to averaging ƒ_{A} and ƒ_{D}, each function is scaled such that the total area under each spectrum is equal to the quantum yield of EYFP or ECFP, respectively. The approximation relies on the fact that the optical transfer functions for the excitation and emission paths of an optical detection system such as a microscope are nearly constant over their respective bandwidths. Averages were calculated from experimentally determined excitation and emission spectra, and the following values were obtained M_{A}=[ε_{A}(λ)]_{80=430-450 nm}; [ƒ_{A}(λ)]_{λ=505-575 nm}=0.036;and M_{D}=[ε_{D}(λ)]_{λ=430-450 nm}·[ƒ_{D}(λ)]_{λ=505-575 nm}=0.058. Substituting Equations A30 and A31 into Equations A28 and A29 then yields the following expressions for N_{A} and N_{D}

YFP_{FRET}(440,535,direct)≈N_{A}·Iₒ·C ·M_{A} [A32]

CFP_{FRET}(440,535,direct)≈N_{D}·Iₒ·C·M_{D}−E_{eff}·YFP_{FRET}(440,535,direct)·M_{D}/M_{A} [A33]

Substituting Equations A32 and A33 into Equation A26 yields an experimentally-based estimate of A_{b}, according to

$$\begin{matrix}{A}_{b} = \frac{\begin{matrix}{CFP}_{EST} + {YFP}_{EST} + {K}_{d , EFF} - \\ \sqrt{\begin{matrix}{( {CFP}_{EST} + {YFP}_{EST} + {K}_{d , EFF} )}^{2} - \\ 4 \cdot {CFP}_{EST} \cdot {YFP}_{EST}\end{matrix}}\end{matrix}}{2 \cdot {YFP}_{EST}} & [ A34 ] \\ {CFP}_{EST} = \frac{{\begin{matrix}{CFP}_{FRET} ( 440 , 535 , direct ) + \\ ( FR - 1 ) [ {ϵ}_{γ FP} ( 440 ) / {ϵ}_{CFP} ( 440 ) ] \cdot \\ {YFP}_{FRET} ( 440 , 535 , direct ) \cdot {M}_{D} / {M}_{A}\end{matrix}}^{6 \; 4 \; 4 \; 4 \; 4 \; 4 \; 4 {7}^{{E}_{eff}} 4 \; 4 \; 4 \; 4 \; 4 \; 4 \; 8}}{{M}_{D}} & [ A35 ] \\ {YFP}_{EST} = \frac{{YFP}_{FRET} ( 440 , 535 , direct )}{{M}_{A}} & [ A36 ]\end{matrix}$$

K_{d,EFF}=2·K_{d}·V·N_{avogadro}·IₒC [A37]

Regression analysis can be used to estimate A_{b} in individual cells. A given cell provides the experimentally determined FRET ratio (FRₑₓₚ) and three 3³-FRET measurements. Upon selecting parameters ΔFRₘₐₓ and K_{d,EFF}, Equation A34 will translate the 3³-RET measurements into a prediction of A_{b}, and Equation A23 will in turn translate the predicted A_{b} into a predicted FR(FR_{predicted}). Parameters ΔFRₘₐₓ and K_{d,EFF} can be adjusted until the squared error (FRₑₓₚ−FR_{predicted})² is minimized.

Minimizing the error for a single cell, in itself, would not be a very stringent constraint on the parameters. However, the same ΔFRₘₐₓ should apply to different cells expressing variable numbers of donor and acceptor molecules. In addition, if the volume of cells (V) is roughly comparable, then the same K_{d,EFF} should apply to different cells. Thus, a single pair of ΔFRₘₐₓ and K_{d,EFF} values can be applied to all cells, and calculate an aggregate squared error (FRₑₓₚ−FR_{predictd})² summed from all cells. ΔFRₘₐₓ and K_{d,EFF} can then be adjusted to minimize the aggregate error over many cells, thus providing a much more stringent constraint on these parameters.

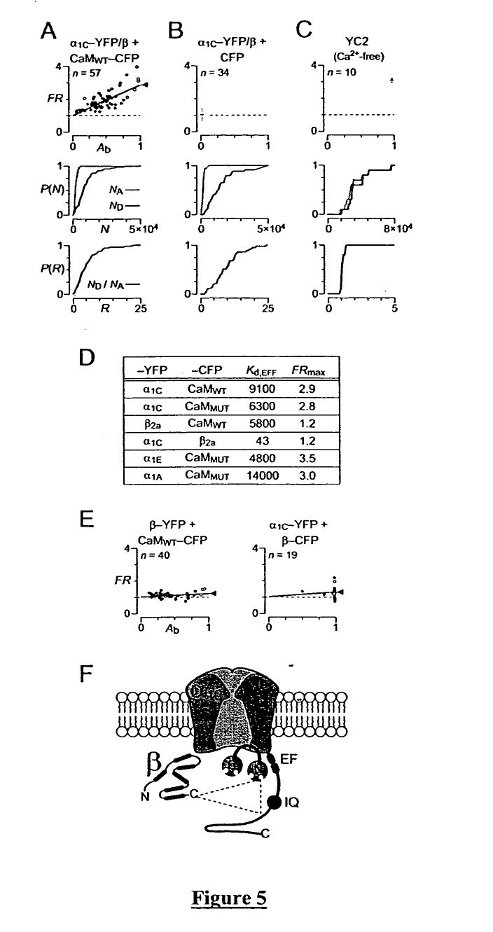

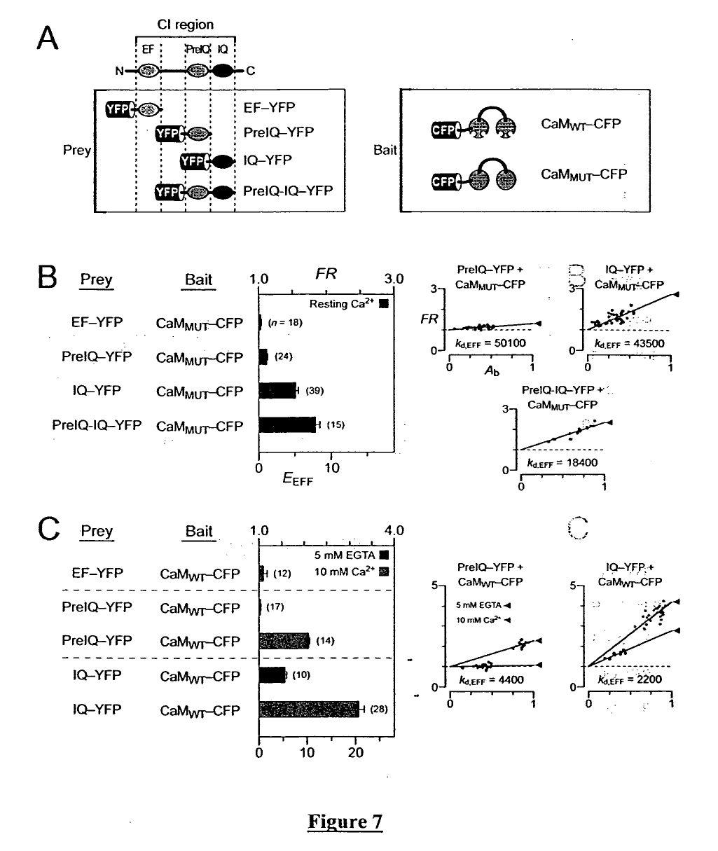

Comparison between data and predicted values are shown in FIG. 5A-B and FIG. 7B-C for various FRET pairs. As can be seen from the Figures, application of the above equations provides an estimate of the relative dissociation constant for binding (K_{d,EFF}) and maximal FR (FRₘₐₓ) when every EYFP-tagged molecule is associated with a ECFP-tagged partner (i.e., when the fraction bound is unity; see, for e.g., FIG. 7A, arrows). A summary of these estimated constants for several FRET pairs is shown in FIG. 5D. The estimates of FRₘₐₓ can be used to calculate inter-fluorophore distances according to the Förster equation (E=1/[1+(R/R₀)⁶]), where the orientation factor κ² has been estimated to be near 2/3 and R₀ has been estimated to be about 49 Å.

This analysis immediately provides several dividends. First, the overall linearity of the FRₑₓₚ versus A_{b} plot, based upon an optimal pair of ΔFRₘₐₓ and K_{d,EFF} values, provides some evidence that donor and acceptor molecules interact via 1:1 saturable binding, although the scatter in the data precludes a definitive interpretation. Second, the estimated ΔFRₘₐₓ value provides a means to estimate the true FRET efficiency (Equation A23), which is required to calculate donor-acceptor distance. Finally, the estimated K_{d,EFF} values determined for molecular interactions provide an indication of relative affinity, even without explicitly determining the relationship to actual K_{d} values (which, in principle, could be done according to Equation A34).

As outlined above, 3³-FRET determination of K_{d,EFF} and FRₘₐₓ is conveniently applied to measurements of FR; however, it may also be applied to many other quantitative FRET indices. Generally, measurements of FRET can be based on the enhancement of acceptor fluorescence emission (as with FR) or on the quenching of donor fluorescence emission. 3³-FRET can be applied exactly as illustrated above to any method based on acceptor emission, provided that the method generates an index that can be linearly related to E. For methods based on donor emission that generate an index related to E (e.g., acceptor photobleaching method), two simple alterations make the method compatible with 3³-FRET. First, an equivalent FR can be computed based on the known relationship between E and FR, as FR_{equiv}=1+E[ε_{CFP}(440)/ε_{YFP}(440)], and FR_{equiv} can be substituted for FR in the description of 3³-FRET above. Second, terms describing the number and/or concentration of donor and acceptor molecules in the field of view (e.g., N_{D} and N_{A}) must be swapped. These changes reflect the fact that a FRET method based on donor fluorescence will generate binding affinities with respect to the donor-tagged molecule, whereas a FRET method based on acceptor fluorescence will generate binding affinities with respect to the acceptor-tagged molecule. See Erickson, et al., 2001, Neuron 31:973-985 for a complete discussion of the differences between donor-based and acceptor-based methods. For all methods, 3³-FRET requires donor and acceptor filter set readings that can be related, as above, to the number of donors and acceptors in the field of view.

Generalization of 3³-FRET Method for Donor:Acceptor Fluorophore Pairings Other than ECFP and EYFP

Although the 3³-FRET method described above has been exemplified using ECFP as the donor fluorophore and EYFP as the acceptor fluorophore, the system can be generalized for other donor:acceptor pairs which meet the following criteria: there is significant overlap between the donor emission spectrum and the acceptor absorption spectrum; the donor:acceptor spectral properties permit use of a filter set that can detect emission predominantly from the donor while predominantly excluding emission from the acceptor; and, donor:acceptor spectral properties permit use of a filter set that can predominantly excite the acceptor while predominantly excluding excitation of the donor. Thus, all the formulations throughout can be applied directly by substituting a suitable donor for ECFP and a suitable acceptor for EYFP. For example, 3³-FRET has been applied to measure FRET for the pairs EGFP/DsRed and ECFP/DsRed (Moon, et al. Biophys. J. 80: 362a.). Other exemplary fluorophores which can be used in FRET assays as donor or acceptor include, but are not limited to, those listed in Table 1, below.

TABLE 1
Fluorophores

Acridine orange (+DNA)
Acridine orange (+RNA)
Alexa Fluor ® 350
Alexa Fluor ® 430
Alexa Fluor ® 488
Alexa Fluor ® 532
Alexa Fluor ® 546
Alexa Fluor ® 568
Alexa Fluor ® 594
Alexa Fluor ® 633
Alexa Fluor ® 647
Alexa Fluor ® 660
Alexa Fluor ® 680
Allphycocyanin
AMCA/AMCA-X
7-Aminoactinomycin D (7-AAD)
7-Amino-4-methylcoumarin
Aniline Blue
ANS
ATTO-TAG ™ CBQCA
ATTO-TAG ™ FQ
Auramine O-Feulgen
BCECF (high pH)
BFP (blue fluorescent protein)
BOBO ™-1, BO-PRO ™-1
BOBO ™-3, BO-PRO ™-3
BODIPY ® FL
BODIPY ® TMR
BODIPY ® TR-X
BODIPY ® 530/550
BODIPY ® 558/568
BODIPY ® 564/570
BODIPY ® 581/591
BODIPY ® 630/650-X
BODIPY ® 650/665-X
BTC
Calcein
Calcein Blue
Calcium Crimson ™
Calcium Green-1 ™
Calcium Orange ™
Calcofluor ® White
5-Carboxyfluorescein (5-FAM)
5-Carboxynapthofluorescein
6-Carboxyrhodamine 6G
5-Carboxytetramethylrhodamine (5-TAMRA)
Carboxy-X-rhodamine (5-ROX)
Cascade Blue ®
Cascade Yellow ™
CCF2 (GeneBLAzer ™)
CFP (Cyan Fluorescent Protein)
Chromomycin A3
Cl-NERF (low pH)
CPM
6-CR 6G
CTC Formazan
Cy2 ®
Cy3 ®
Cy3.5 ®
Cy5 ®
Cy5.5 ®
Cy7 ®
Dansyl cadaverine
Dansylchloride
DAPI
Dapoxyl ®
DiA (4-Di-16-ASP)
DiD (DilC₁₈(5))
DIDS
Dil (DilC₁₈(3))
DiO (DiOC₁₈(3))
DiR (DilC₁₈(7))
Di-4 ANEPPS
Di-8 ANEPPS
DM-NERF (4.5-6.5 pH)
DsRed (Red Fluorescent Protein)
ELF ®-97 alchol
EBFP (enhanced blue fluorescent protein)
ECFP (enhanced cyan fluorescent protein)
EGFP (enhanced green fluorescent protein)
EYFP (enhanced yellow fluorescent protein)
Eosin
Erythrosin
Ethidium bromide
Ethidium homodimer-1 (EthD-1)
Europium (III) Chloride
5-FAM (5-Carboxyfluorescein)
Fast Blue
Fluorescein (FITC)
Fluo-3
Fluo-4
FluorX ®
Fluoro-Gold ™ (high pH)
Fluoro-Gold ™ (low pH)
Fluoro-Jade
FM ® 1-43
Fura-2 (high calcium)
Fura-2/BCECF
Fura Red ™ (high calcium)
Fura Red ™ /Fluo-3
GeneBLAzer ™ (CCF2)
GFP Red Shifted (rsGFP)
GFP Wild Type, UV excitation
GFP Wild Type, non-UV excitation
Hoechst 33342 & 33258
7-Hydroxy-4-methylcoumarin (pH 9)
1,5-IAEDANS
Indo-1 (high calcium)
Indodicarbocyanine
Indotricarbocyanine
JC-1
6-JOE
JOJO ™-1, JO-PRO ™-1
Lissamine rhodamine B
LOLO ™-1, LO-PRO ™-1
Lucifer Yellow
LysoSensor ™ Blue (pH 5)
LysoSensor ™ Green (pH 5)
LysoSensor ™ Yellow/Blue (pH 4.2)
LysoTracker ® Green
LysoTracker ® Red
LysoTracker ® Yellow
Mag-Fura-2
Mag-Indo-1
Magnesium Green ™
Marina Blue ®
4-Methylumbellierone
Mithramycin
MitoTracker ® Green
MitoTracker ® Orange
MitoTracker ® Red
NBD (amine)
Nile Red
Oregon Green ® 488
Oregon Green ® 500
Oregon Green ® 514
Pacific Blue ™
PBFI
C-phycocyanin
R-phycocyanin
R-phycoeythrin (PE)
PKH26
POPO ™-1, PO-PRO ™-1
POPO ™-3, PO-PRO ™-3
Propidium Iodide
PyMPO
Pyrene
Pyronin Y
Quinacrine Mustard
Resorufin
Red Fluorescent Protein (DsRed)
RH 414
Rhod-2
Rhodamine B
Rhodamine Green ™
Rhodamine Red ™
Rhodamine Phalloidin
Rhodamine 110
Rhodamine 123
5-ROX (carboxy-X-rhodamine)
SBFI
SITS
SNAFL ®-1 (high pH)
SNAFL ®-2
SNARF ®-1 (high pH)
Sodium Green ™
SpectrumAqua ®
SpectrumGreen ® #1
SpectrumGreen ® #2
SpectrumOrange ®
SpectrumRed ®
SYTO ® 11
SYTO ® 13
SYTO ® 17
SYTO ® 45
SYTOX ® Blue
SYTOX ® Green
SYTOX ® Orange
5-TAMRA (5-Carboxytetramethylrhodamine)
Tetramethylrhodamine (TRITC)
Texas Red ®/Texas Red ®-X
Thiacarbocyanine
TOTO ®-1, TO-PRO ®-1
TOTO ®-3, TO-PRO ®-3
TO-PRO ®-5
WW 781
X-Rhodamine (XRITC)
YFP (Yellow fluorescent Protein)
YOYO ®-1, YO-PRO ®-1
YOYO ®-3, YO-PRO ®-3

Preferably, the fluorophores are peptides or polypeptides (e.g., such as GFP-related proteins) which can be fused to a polypeptide(s) of interest. Sequences of GFP-related proteins are described in U.S. Pat. No. 5,625,048; U.S. Pat. No. 5,77,079; U.S. Pat. No. 6,306,600; U.S. Pat. No. 6,251,384; U.S. Pat. No. 6,235,968; U.S. Pat. Nos. 6,232,523; 6,130,313; U.S. Pat. No. 6,090,919; U.S. Pat. No. 6,020,192; U.S. Pat. No. 6,054,387; and U.S. Pat. No. 5,804,387; for example, the entireties of which are incorporated herein by reference.

Systems for Performing 3³-FRET

A number of optical systems can be used to detect FRET between a donor and acceptor molecule such as ECFP and EYFP. In one aspect, the invention provides a system optimized for performing 3³-FRET. To accomplish this 3³-FRET process, three filter sets are sequentially placed between the light source and the specimen, and between the specimen and the detector. The individual filter sets each comprise a filter between the light source and the specimen and a filter between the specimen and the detector. Each filter set transmits and/or reflects specific wavelengths of light. In the first filter set (“donor filter set”), the filter between the light source and specimen maximally transmits a wavelength of light that excites the donor (and possibly the acceptor), and the filter between the specimen and the detector maximally transmits wavelengths of light where only the donor emits photons. In the second filter set (“acceptor filter set”), the filter between the light source and specimen maximally transmits a wavelength of light that preferentially excites the acceptor, and the filter between the specimen and the detector maximally transmits wavelengths of light where mainly the acceptor emits photons (and possibly the donor emits photons). In the third filter set (“FRET filter set”), the filter between the light source and specimen maximally transmits a wavelength of light that excites the donor (and possibly the acceptor), and the filter between the specimen and the detector maximally transmits wavelengths of light where mainly the acceptor emits photons (and possibly the donor emits photons).

The 3³-FRET method processes these three light intensity readings, each obtained with a different filter set engaged, and yields a quantitative readout of the strength of FRET interaction, termed “the FRET ratio” or FR. FR furnishes the fractional increase in acceptor fluorescence due to FRET.

Preferably, three filter cubes comprise the first, second, and third filter sets. Preferably, each filter cube contains an excitation filter, a dichroic mirror, and an emission filter. Preferably, the excitation filter is a band pass or high pass filter that allows only short wavelength light from a light source to pass through. Also preferably, the emission filter is a band pass or low pass filter that passes only long wavelength light emitted by the object in response to illumination by the shorter wavelength exciting light. The dichroic mirror is a beam splitter that reflects the excitation light onto a specimen, e.g., such as a cell, and then allows emitted light from the specimen to pass through. The “cut on” wavelength of the dichroic mirror generally lies between the transmission bands of the excitation filter and the emission filter.

In a particularly preferred aspect, 3³-FRET filter-cubes used comprise a ECFP cube comprising an excitation filter of D440/20M, a dichroic mirror of 455DCLP, and an emission filter of D480/30M (available commercially from Chroma, Inc., Brattleboro, Vt.; a EYFP cube comprising an excitation filter of 500DF25, a dichroic mirror of 535DRLP, and an emission filter of 530EFLP; and a FRET filter cube comprising an excitation filter of 440DF20, a dichroic mirror of 455DRLP, and an emission filter of 535DF25 (EYFP cubes and FRET cubes are obtainable from Omega Optical). The numerical designators in each case refers to the peak wavelength of light transmitted by each filter. For example, an excitation filter of D440/20M, transmits light maximally at 440 nm, with a 20 nm bandwidth. LP signifies a longpass filter. Other types of filter sets and cubes can be used, and the examples above are non-limiting. For example, in one aspect, filter cubes are precisely machined as described in PCT/US98/11,390 9,855,026 to minimize overlap in the emission spectra between donor and acceptor molecules.

The system further comprises a light source and also can comprise one or more optical fibers for transmitting light to a specimen (e.g., such as a cell). In order to generate enough excitation light intensity to furnish secondary fluorescence emission capable of detection, a powerful light source generally is preferred, such as a mercury or xenon arc (burner) lamp, for producing high-intensity illumination powerful enough to image faintly visible fluorescence specimens. A laser light source (e.g., a gas laser such as a nitrogen, helium, neon, or argon laser; uv laser; semi-conductor laser; pulsed laser, solid-state diode laser, and the like) also can be used and, in one aspect, a scanning mechanism is provided for moving the light source relative to the sample so that light can be scanned across the specimen (e.g., for obtaining three-dimensional images).

Preferably, the cubes are connectable to an appropriate detection device which can comprise, but is not limited to: one or more photodetectors, a filter, a CCD camera, a streak tube, an endoscopic imaging system, an endoscopic fluorescence imaging microscope, a fiber optic fluorescence imaging microscope, a computer used in the fluorescence analysis, and the like. Preferably, the system also comprises a holder for holding at least one of the cubes in position relative to a specimen, the light source, and the detector, so that light from the light source can be received by the specimen and light emitted by the specimen can be received by the detector.

In one aspect, each filter cube can be sequentially positioned (e.g., via a holder slideable in a horizontal plane), relative to a light source and light detector to obtain sequential light intensity readings. In one aspect, when a filter cube is so positioned, the cube is rotatable about a vertical axis for selectively aligning an optical path with a light source and one or more focusing lens. The system also can include a wavelength divider such as a filter, prism, diffraction grating, or image-subtracting double monochromator.

The system further can comprise a sample support, e.g., such as a stage, and a scanning mechanism for scanning the support relative to both the light source and the detection device. Scanning can be mechanical or automated. Preferably, at least a portion of the sample support is optically transmissive.

The system also can include an image processor and/or an image display device. The image processor may be a suitably programmed personal computer, while the image display device may be a computer monitor (e.g., CRT or LCD display) or a printer. For example operation, an image of an illuminated sample can be obtained by the detector device and input into the processor as a digitized pixel image. A set of three such images from each channel (donor, acceptor, and FRET) can be processed as three spatially coregistered images or can be treated as single images in which each pixel has three color space coordinates corresponding to the monochrome wavelengths. Preferably, the processor comprises software for implementing 3³-FRET analysis. A flowchart of the process that such software would implement is shown in FIG. 8.

The optical detection system can include, but is not limited to: an epifluorescent microscope, a 3D imaging system, such as a confocal microscope (single-photon confocal microscope) or a two-photon microscope. In addition to permitting subcellular monitoring, such the latter two systems would facilitate the identification of molecular interactions (e.g., such as protein-protein interactions) deep in living tissue samples.

In one aspect, the optical system is a flow cytometer comprising a dropping nozzle through which individual cells can be passed in a single small droplet of suspending media (e.g., a buffer or cell culture media). At least one coherent light source (e.g., a laser) is placed in optical proximity to the droplet to excite fluorescence in the cell. Light emitted by the cell is channeled into a light path using a least one focusing element which is separated into various wavelengths using at least three dichroic mirrors which divert light into each of at least three filters: a donor filter, an acceptor filter, and a FRET filter. Light transmitted through the filters are detected using separate detector devices (e.g., such as photomultipliers). Signals from the photomultipliers are sent to a processor which performs 3³-FRET computations to calculate FRET.

In one aspect, droplets with particular fluorescent characteristics (e.g., reflecting interacting donor:acceptor pairs) are given an electric charge. Charged and uncharged droplets are separated as they fall between charged plates. Thus, the system can be used to both evaluate molecular interactions as well as to identify and sort cell populations in which donor acceptor interactions have or have not occurred.

In a further aspect, the optical system is a plate reader. Such a plate reader can be coupled to a robotic fluid transfer system to maximize assay throughput.

3³-FRET Assays

The 3³-FRET assays described herein are generally nondestructive of cells, as compared, for example, to the acceptor bleaching method of Miyawaki and Tsien, 2000, supra. The assays are also rapid, facilitating high throughput screening (HTS) of specimens. For example, a cell-based assay according to the invention can be performed in 3-5 minutes using an epifluorescence microscope. HTS screens also can be performed using FACs sorting machines, making it possible to evaluate responses of single cells in under 5 minutes, and even within the time-of-flight requirements of these machines (i.e., within seconds). This contrasts with the bleaching method of Miyawaki and Tsien, 2000, supra, which take minutes, precluding its use in a FACS sorting machine.

3³-FRET can be used to monitor the responses of a FRET-based sensor in analyte detection assays. For example, a donor tagged molecule and an acceptor tagged molecule can be bound to a binding protein that changes its conformation upon binding to an analyte (see, e.g., as described in U.S. Pat. No. 6,197,928). The change in conformation leads to a change in the relative position and orientation of the donor and acceptor molecules and FRET. The binding protein can be in solution or immobilized on a solid phase (e.g., a particle, microparticle, bead, microbead, sphere, magnetized particle, capillary, slide, wafer, cube, membrane, filter, and the like), creating a FRET-based sensor for the analyte. The degree of FRET can be correlated with the concentration of analyte in the sample. In one aspect, the degree of FRET is determined over different time periods to determine changes in the concentration of an analyte in the sample.

Preferably, the donor molecule is ECFP while the acceptor molecule is EYFP. Suitable binding proteins which change conformation upon binding to an analyte include, but are not limited to, calmodulin (CaM), cGMP-dependent protein kinase, steroid hormone receptors (or ligand binding domains thereof), protein kinase C, inositol-1,4,5-triphosphate receptor, alphachymotrypsin, or recoverin (see, e.g., as described in Katzenellenbogen and Katzenellenbogen, 1996, Chemistry & Biology 3: 529-536; Ames, et al., Curr. Opin. Struct. Biol. 6: 432-438; U.S. Pat. No. 5,254,477). In one aspect, the binding protein is also responsive to an intracellular signaling molecule (e.g., such as Ca²⁺) (see, e.g., Falke, et al., 1994, Quart. Rev. Biophys. 27: 219-290). Other suitable signaling molecules include, but are not limited to, the calmodulin-binding domain of M13, smMLCKp, CaMKII, Caldesmon, Calspermin, Calcineurin, PhK5, PhK13, C28W, 59-kDa PDE, 60-kDa PDE, NO-30, AC-28, Bordetella pertussis AC, Neuro-modulin, Spectrin, MARCKS, F52, [beta]-Adducin, HSP90a, HIV-1 gp160, BBMHBI, Dilute MHC, Mastoparan, Melittin, Glucagon, Secretin, VIP, GIP, or Model Peptide CBP2. The binding of these signaling molecules also can be monitored by monitoring changes in the interactions between the binding protein and the analyte.

In another aspect, the binding protein is an enzyme and FRET is an indication of substrate catalysis as well as binding (see, e.g., as described in U.S. Pat. No. 5,254,477). In a further aspect, a donor and acceptor molecule are held together by a cleavable linker, e.g., such as a peptide linker comprising a cleavage site for cleaving molecule. While in their linked state, FRET occurs between the donor and acceptor molecule; however, upon cleavage by a cleaving molecule (e.g., such as an enzyme), the donor and acceptor molecule are separated resulting in a decrease in FRET. In this embodiment, therefore a decrease in FRET is used as a measure of an analyte in a sample. In one aspect, the assay is used to detect an intracellular protease. Suitable linkers comprising cleavage sites are described in U.S. Pat. No. 5,981,200, for example.

The donor/acceptor tagged binding protein can be generated using methods routine in the art and as described in U.S. Pat. No. 6,197,928, for example, the entirety of which is incorporated by reference herein. Sequences for both ECFP and EYFP are known in the art, as are sequences for the coding regions of the binding proteins exemplified above. It is contemplated that additional coding sequences for binding proteins will become known and the examples provided herein are non-limiting.

In one aspect, the donor molecule and acceptor molecule are linked to the binding protein using a suitable linker for maintaining the donor and acceptor molecule greater than 100 Å away from each other when the tagged binding protein is in a solution or immobilized on a substrate, and less than 100 Å when the tagged binding protein is bound to an analyte. In order to optimize the FRET effect, the average distance between the donor and acceptor molecules is between about 1 nm and about 10 nm, preferably between about 1 nm and about 6 nm, and more preferably between about 1 nm and about 4 nm, when the analyte is bound (or released). In one aspect, the linker comprises between about one and 30 amino acid residues in length, preferably between about two and 15 amino acid residues. One preferred linker moiety is a -Gly-Gly-linker. One preferred linker moiety is a linker comprising a plurality of serines and glycines. Preferably, such a linker is about 50% serine. Flexible linker molecules and constraints on the design of linker molecules are known in the art and are described in U.S. Pat. No. 6,197,928; U.S. Pat. No. 5,254,477; Huston, et al., 1988, Proc. Natl. Acad. Sci. USA 85: 5879-5883; Whitlow, et al., 1993, Protein Engineering 6: 989-995 (1993); and Newton, et al., 1996, Biochemistry 35: 545-553. Where the donor and acceptor molecules are not peptides or polypeptides, they can be conjugated to the binding protein using chemical conjugation methods as are well known in the art.

The sensor also can be used to sense molecules in an intracellular environment. For example, the tagged binding protein can be introduced into a cell and changes in the proximity of donor and acceptor molecules upon binding of an intracellular molecule binding to the binding protein can be detected using 3³-FRET and the optical system as described above.

In one aspect, the tagged binding protein comprises a localization signal to facilitate introduction of the sensor into the cell and/or to target the sensor to a particular intracellular compartment. Suitable localization sequences include, but are not limited to: a nuclear localization sequence, an endoplasmic reticulum localization sequence, a peroxisome localization sequence, a mitochondrial localization sequence, and a peroxisome localization sequence. Additional localization sequences are described in U.S. Pat. No. 6,197,928 and in Stryer, 1995, Biochemistry (4th ed.). W. H. Freeman, Ch. 35, for example. In another aspect, cells are electroporated to transiently introduce pores into the cells to facilitate uptake of the tagged binding protein.

In a further aspect, donor and acceptor pair interactions are used to detect and or quantitate a nucleic acid analyte. A first and second oligonucleotide probe can be labeled with a donor and acceptor molecule, respectively, for example, by chemical conjugation. The sequence of the first probe is selected to be complementary to a first portion of a target sequence while the sequence of a second probe is selected to be complementary to a second portion of the target sequence, such that hybridization of the first and second probe to the hybridization sequence brings the donor and acceptor molecule in sufficient proximity to each other to cause FRET (see, e.g., as described in Wittwer, et al., 1997, Biotechniques 22: 130-138; Bernard, et al., 1998, Am. J. Pathol. 153: 1055-1061). Mismatches caused by polymorphisms such as SNPs that disrupt the binding of either of the probes can be used to detect mutant sequences present in a DNA sample.

In a preferred aspect, the first and second oligonucleotides are introduced into a cell using methods routine in the art (e.g., transfection, transformation, electroporation, microinjection) and FRET is detected using 3³-FRET and the optical system as described above. In still another aspect, a nucleic acid substrate is provided for measuring DNA-polypeptide interactions using FRET. Preferably, the substrate is linked to a donor and acceptor (e.g., by chemical conjugation) in such a way that the donor and acceptor are less than 100 Å apart. The nucleic acid substrate is incubated with a sample and binding of a polypeptide increases the distance between the donor and acceptor molecule, i.e., decreasing FRET. In one aspect, the polypeptide is a nucleic acid cleaving enzyme, such as a nuclease. Preferably, the nucleic acid substrate is immobilized on a substrate (e.g., such as a glass slide) and FRET is detected using 3³-FRET and the optical system as described above.

It should be obvious to those of skill in the art that many analyte detection assays using FRET are possible, and that modifications to these assays to perform 3³-FRET is within the skill of the art using the invention described herein, and is encompassed within the scope of the present invention.

Some donor acceptor pair interactions are susceptible to pH, such that FRET changes upon a change in the pH of a solution surrounding the donor acceptor molecules. For example, the absorption of the basic form of phenol red rises with increased pH and overlaps the emission spectrum of eosin, resulting in increased FRET as pH is raised from 6 to 10. A change in pH can thus be monitored by monitoring changes in FRET.

Therefore, in one aspect, FRET sensors are generated by immobilizing appropriate donor acceptor pairs on a substrate (e.g., a polymer) at suitable distances using linker molecules. Suitable fluorophore pairs that can be used and their excitation and emission wavelength(s) are described in U.S. Pat. No.5,254,477, the entirety of which is incorporated by reference herein. Changes in FRET can be detected readily using the optical system and 3³-FRET methods described above.

Stable cells lines expressing a known interacting pair of donor and acceptor-tagged molecules can be used in HTS assays to screen for modulators of these molecules, such as drugs. In one aspect, a screen for compounds that disrupt the protein-protein interactions, is performed. Such a screen can be made high throughput by robotic application of different compounds to cultures of cells in multi-well plates. A custom plate reader designed to perform 3³-FRET on each of the wells can be used to rapidly identify candidate compounds that inhibit the protein-protein interaction of interest. Plate readers need only be modified to allow engagement of three filter sets, as described above under optical systems.

In one aspect, first and second molecules (e.g., nucleic acids and/or proteins) are tagged with donor and acceptor molecules (e.g., by chemical conjugation or by genetic engineering). Interaction between the first and second molecules brings the donor and acceptor molecules sufficiently close together to cause FRET. The first and second molecules are introduced into a cell using methods known in the art (e.g., transfection, transformation, electroporation, microinjection) and the cell is contacted with a sample suspected of comprising a modulator of the interaction. Suitable interacting molecules include, but are not limited to, ligands and receptors; antibodies and antigens; calmodulin and calcium; G proteins, GTP and G-Protein Coupled Receptors; and the like.

FRET in the cell is detected using 3³-FRET, for example, with the optical system described above. The strength of FRET is compared to a baseline, e.g., the amount of FRET in the cell prior to exposure to the sample, or the strength of FRET in a substantially identical cell into which the first and second molecules have been introduced but which has not been exposed to sample. A modulator is identified as a compound which produces a significant change with respect to the baseline FR, using routine statistical methods. As used herein, a “substantially identical cell” refers to a genetically identical cell.

In one aspect, the method further includes the step of contacting the cell with a compound at a first time and a second time, and measuring a change in FRET at the first time and the second time.

In another aspect, the method further includes the step of contacting a cell with a first concentration of a compound, and a substantially identical cell with a second, different concentration and determining FRET after each contacting to determine a dose-response curve for the compound.

In a further aspect, a donor and acceptor tagged molecules are provided as part of a two-hybrid system to identify molecules which interact with a polypeptide of interest (see, e.g., Fields and Song, 1989, Nature 340: 245-246; WO 94/10300; U.S. Pat. No. 5,283,173). For example, a bait protein can be generated by fusing the polypeptide of interest to a donor polypeptidepeptide (e.g., such as ECFP), while prey proteins can be generated from random sequences fused to an acceptor peptide (e.g., such as EYFP). Interactions between the polypeptide of interest and the bait protein can be identified by the FRET which occurs as donor and acceptor polypeptides are brought in sufficient proximity. 3³-FRET analysis of bait and prey interactions would provide for a high-throughput discovery strategy, since protein-protein interactions are almost instantaneously detected by 3³-FRET (e.g., as compared to systems such as yeast two-hybrid systems). Single-cell rescue of nucleic acid sequences encoding an interacting prey polypeptide can be used to specify the identity of the interacting prey polypeptide. In this manner, discovery of unknown interaction partners with a specified bait polypeptide can be determined. For example, the assay can be used to identify ligands for orphan receptors. Application of this approach to many cells in parallel, such as using plate-reader technology and robotic fluid transfer systems (e.g., facilitating minipreps of samples), permits high-throughput identification of interacting molecules. In a particularly preferred aspect, the assay can be used to identify interacting molecules in living mammalian cells.

The assays above can be used to provide clinical tests (e.g., diagnostic and prognostic assays), as well as screening assays. For example, a cellular process or condition can be diagnosed by performing the analyte detection assays described above to detect a marker of a disease (e.g., such as a tumor-specific antigen). Alternatively, 3³-FRET can be used to screen for altered molecular interactions that are known to be perturbed during the cellular process or condition. Thus, a specimen can be obtained from a patient suspected of having, or at risk for developing a disease and can be evaluated for the presence of an analyte or altered interaction by using 3³ FRET, after introduction of a suitable FRET-based biosensor into the patient specimen. The measure of FRET obtained from the specimen can then be compared to a measure obtained from a control, such as a normal patient.

Because the assays can be performed in living cells, the effect of a test compound, such as a drug on the expression of the analyte/molecular interaction can be evaluated over time to examine the effect of the drug on the normalization of a physiological response. In addition to amount of FRET, the localization of FRET also can be monitored. For such cell-based assays, the specimen can be place on a sample holder comprising a culture medium. Various parameters of the culture medium can be regulated, such as pH and temperature, using automated controls (e.g., sensors and tubing systems which can deliver appropriate reagents to the culture medium in response to conditions sensed by the sensors). Using a FACs sorting system as described above, cells comprising analytes, or in which molecular interactions have occurred, can be identified and sorted because of their unique spectral properties.

Physical distances between molecular landmarks can be calculated in order to characterize a protein-protein interaction (see, e.g., Stryer and Haugland, Proc. Natl. Acad. Sci. USA 58: 719-726). For example, FR readings significantly greater than 1 can only result from donor acceptor molecules tagging two proteins or protein domains separated by less than about 100 Å (e.g., well within the characteristic dimensions of a Ca²⁺ channel complex). Thus, 3³ FRET can be used to model the position of binding sites in complexed proteins. An example of such a method is described further in Example 1 and in FIGS. 5A-F.

### EXAMPLES

The invention will now be further illustrated with reference to the following examples. It will be appreciated that what follows is by way of example only and that modifications to detail may be made while still falling within the scope of the invention.

### Example 1

### Application of 3³-FRET to Reveal Preassociation of Calmodulin with Voltage-Gated Ca²⁺ Channels in Single Living Cells

Voltage-gated Ca²⁺ channels trigger essential physiological processes, including contraction, secretion and expression. Among the most intriguing forms of Ca²⁺ channel modulation are the feedback regulation of L-type (α_{1C}) and P/Q-type (α_{1A}) channels by intracellular Ca²⁺ fluctuations, acting in an unconventional channel-calmodulin (CaM) interaction. In particular, Ca²⁺-insensitive mutant CaM (CaM_{MUT}) eliminates Ca²⁺ dependent modulation in both channel types, hinting that CaM may be “preassociated” with these channel complexes even before channel opening, so as to enhance detection of local Ca²⁺. Though compelling, in vitro experiments testing this model have provided conflicting results.

3³-FRET was used to probe constitutive associations between Ca²⁺ channel subunits and CaM in single living cells, using variants of the green fluorescent protein (GFP) as fluorophore tags. This rapid, non-destructive assay detects steady-state associations between CaM (or CaM_{MUT}) and the pore-forming α₁ subunit of L-type, P/Q-type, and surprisingly, R-type (α_{1E}) Ca²⁺ channels. Moreover, the assay was used to map a triangle formed by three key channel landmarks: the α₁ subunit, the auxiliary β₂ₐ subunit, and CaM. These results mark the first direct evidence for binding of CaM to calcium channel complexes in resting cells and underscore the utility of 3³-FRET for probing protein-protein interactions in living systems.

An unusual twist to the classic view of calmodulin is that apoCaM sometimes preassociates with a target molecule, whose activity is subsequently modulated as Ca²⁺-CaM shifts to a different site on the target. This arrangement is a potent means of ensuring selective responsiveness to local Ca²⁺ and, in the case of Ca²⁺ channels, of permitting accelerated modulation initiated by local Ca²⁺ influx. Although there are relatively few instances where traditional in vitro biochemistry confirms such preassociation, the actual prevalence of this mechanism may be far greater, especially for ion channels whose potential apoCaM interaction might be disrupted by detergents required to solubilize channels for in vitro biochemistry. Assays based on FRET between GFP color mutants ECFP and EYFP obviate such limitations and detect apoCaM interaction in the setting of ultimate relevance, living cells. When excited by short-blue light (440 nm), mixtures of ECFP and EYFP expressed in cells mainly fluoresce at cyan wavelengths, owing to preferential direct excitation of ECFP. However, if ECFP and EYFP are fused to CaM and a target protein, then apoCaM preassociation brings CFP and EYFP within 100, resulting in nonradiative energy transfer (FRET) to EYFP and its ensuing sensitized yellow fluorescence emission. This method provides a useful platform for HTS screening of potential apoCaM-target interactions.

ECFP/EYFP tagged proteins were generated as described below and assayed to verify that resulting fusion proteins preserved the functions and interactions of the tagged proteins. Focusing on L-type (α_{1C}) channels, the functional modulation produced by CaM-channel interaction is feedback inhibition of channel opening by elevated intracellular Ca²⁺ (Ca²⁺-dependent inactivation). Two CaM-channel interactions are believed to underlie such inactivation: (1) Ca²⁺-CaM binding to an “IQ-like” domain on the proximal α_{1C} carboxyl tail (FIG. 1A), which initiates Ca²⁺-dependent inactivation; and (2) inferred preassociation of the Ca²⁺-free form of this CaM with the channel complex, at a presently uncertain site. In designing the fusion of EYFP to the channel, it was theorized that if apoCaM indeed preassociates, it would be close to the IQ site. The α_{1C} carboxyl tail was therefore truncated just beyond the IQ site before fusion to EYFP (see, e.g., FIG. 1A, α_{1C}-EYFP), so as to favor FRET detection of apoCaM interaction. ECFP was fused to the amino lobe of CaM and CaM_{MUT}, yielding CaM_{WT}-ECFP and CaM_{MUT}-ECFP.

HEK293 cells were thinly plated into 3.5-cm culture dishes with No. 0 glass cover slip bottoms (MatTek Corp.) optimized for inverted microscopes. Cells were transiently transfected with FuGene 6 as a means of optimizing transfection using the manufacturer's standard protocol (Roche Molecular Biochemicals) and three days later assessed optically. Just prior to beginning an experiment, the cells were washed twice then bathed in 2 mM CaCl HEPES buffered Tyrodes solution (in mM: CaCl, 2; NaCl, 138; KCl, 4; MgCl₂6H₂O, 1; NaH2PO4H₂O, 0.33; HEPES, 10; pH 7.35 and osmoles adjusted to 300-mOsm with glucose).

Individual cells were visualized with a 40x oil immersion objective on a Nikon TE300 Eclipse inverted microscope. Excitation light was delivered by a 150-Watt short-gap Xenon arc-lamp (Optiquip), gated by a computer-controlled shutter (Uniblitz; Vincent Assoc.) Epi-fluorescence emission light was directed through the side-port into a dual-wavelength detection system adapted from a commercially available indo-I ratio fluoremeter (Univ. of Pennsylvania Biomedical Instrumentation Group). The sideport optical train includes an adjustable aperture in the image plane to clip spurious light from neighboring cells or other background sources, a selectable eyepiece for precise adjustment of image position and focus, an optional beam-splitter, and two 30-mm EMI 9124B (Electron Tubes Limited, England) ambient temperature photon-counting photomultiplier tubes (PMTs).

PMT signals were conditioned by pre-amplifiers, integrated and filtered (at 10 kHz) in the dual-channel fluorometer, and digitized with an ITC-18 programmable data acquisition board (Instrutech Corp.). Shutter control, data acquisition, and automatic dark-current subtraction were managed by custom software combining MATLAB (The MathWorks, Inc.) and C programs which communicate with the ITC-18 using a set of commercial drivers (DeviceAccess, Bruxton Corp.). To minimize measurement variance, 100,000 samples acquired over 0.5 seconds are averaged for each data point.

To correct for autofluorescence and background light scatter, 3³-FRET measurements with gains matching those used in the experiments were applied to single cells expressing untagged channel, CaM and accessory proteins. Background values averaged over many cells are subtracted from the experimental values for each of the 3³-FRET measurements. In practice, HEK293 cells have uniform dimensions, and the background signals on any given day vary little.

Based on measurements of peak extinction coefficients, values of 2.35 mM⁻¹ cm⁻¹, 25.1 mM⁻¹cm⁻¹ and 0.0936. respectively, were used for ε_{YFP}, ε_{CFP} and ε_{YFP}/ε_{CFP}. Efficiencies E (FIGS. 2-4) were calculated from FR according to the equation FR=1+[ε_{CFP}/ε_{YFP}] E, which assumes a one-to-one relationship between the donor and acceptor (see, Equation A23).

The detailed specification of the three optical cubes used were:

| | Excitation | Dichroic | Emission | |
|---|---|---|---|---|
| Cube | Filter | Mirror | Filter | Company |
| ECFP | D440/20M | 455DCLP | D480/30M | Chroma |
| | 440 ± 10 | | 480 ± 15 | |
| EYFP | 500DF25 | 525DRLP | 530EFLP | Omega |
| | 500 ± 12.5 | | | |
| FRET | 440DF20 | 455DRLP | 535DF25 | Omega |
| | 440 ± 10 | | 535 ± 12.5 | |

The conversion ratios used in the 3³-FRET method are as summarized below, for the various tagged constructs. These ratios must be determined for each optical system on which 3³-FRET is applied, as no two systems are exactly alike.

| | n | R_{A1} | R_{A2} |
|---|---|---|---|
| EYFP | 15 | 0.0311 ± 0.0005 | 0.0013 ± 0.0010 |
| α_{1C}-EYFP | 30 | 0.0344 ± 0.0008 | 0.0008 ± 0.0009 |
| α_{1E}-EYFP | 15 | 0.0350 ± 0.0009 | 0.0009 ± 0.0004 |
| α_{1A}-EYFP | 8 | 0.0355 ± 0.0013 | 0.0007 ± 0.0002 |
| B₂ₐ-EYFP | 15 | 0.0338 ± 0.0018 | 0.0012 ± 0.0006 |
| | n | R_{D1} | R_{D2} |
| ECFP | 30 | 0.2090 ± 0.0006 | 0.0036 ± 0.0002 |
| CaM-ECFP | 19 | 0.2082 ± 0.0006 | 0.0067 ± 0.0007 |

The level of CaM expression was qualitatively evaluated by immunostaining to determine by the level of expression of CaM-ECFP, CaM_{MUT} and HEK293 cell endogenous CaM. Three days following transient transfection with calcium-phosphate precipitation, HEK293 cells were scraped from a 10-cm plate, washed with PBS, pelleted and lysed in a small volume of lysis buffer (1% NP40, 20 mM Tris [pH 7.4], 150 mM NaCl) with protease inhibitor cocktail (Complete; Roche). Proteins in the lysates were separated by SDS-polyacrylamide gel electrophoresis (SDS-PAGE) and transferred to a hydrophobic membrane (Immobilon-PSQ; Millipore). Mouse anti-CaM (Research Diagnostics, Inc.) and secondary anti-mouse with conjugated horseradish peroxidase (Amersham) were used in immunoblotting assays and bands were visualized with enhanced chemiluminescence (ECL; Amersham) to determine the presence and relative amounts of the approximately 45 kD CaM fusion proteins and the approximately 20 kD CaM.

As shown in FIGS. 1B-E, the fusion constructs preserved the functional properties of Ca²⁺-dependent inactivation, as well its underlying CaM-channel interactions. HEK293 cells expressing labelled L-type channels (α_{1C} EYFP/β₂ₐ/α₂γ displayed a distinct fluorescent ring at the cell perimeter (FIG. 1B) and had substantial recombinant currents (not shown), confirming that labelled channels are functional and properly target to the plasma membrane. Western blots (FIG. 1C), taken from HEK293 cells transfected with CaM_{WT}-ECFP or CaM_{MUT}-ECFP, showed strong expression of labelled CaMs and no cleavage of linked ECFP. Coexpression of CaM_{WT}-ECFP with α_{1C}-EYFP/β₂ₐ/α₂δ resulted in whole-cell currents with robust Ca²⁺-dependent inactivation (FIG. 1C), as the sharp decay of Ca²⁺ current shows (gray trace). The corresponding Ba²⁺, current (black trace) inactivated little, as expected from the high selectivity of CaM for Ca²⁺ over Ba²⁺.

Averages from multiple cells verified uniformly strong Ca²⁺-dependent inactivation (FIG. 1C, lower), as gauged by the fraction of peak current remaining at the end of 300-ms voltage steps (r₃₀₀). The difference between Ca²⁺ and Ba²⁺ relations (f) quantifies pure Ca²⁺-dependent inactivation. These results closely matched those for unlabelled channels (α_{1C}/(β₂ₐ/α₂δ), indicating that labelled constructs preserved Ca²⁺-dependent inactivation and, by inference, the underlying Ca²⁺-CaM/IQ interaction. In contrast, coexpressing ECFP-tagged CaM_{MUT} (CaM_{MUT}-ECFP), which mimics apoCaM, with labelled L-type channels (α_{1C}/(β₂ₐ/α₂δ) ablated Ca²⁺-dependent inactivation (FIG. 1D), matching results for coexpression of untagged CaM_{MUT} and channels. Importantly, the elimination of inactivation by CaM_{MUT}-ECFP was not due to down-regulation of endogenous CaM (˜18 kD band, FIG. 1C), which was unchanged with overexpression of fusion CaMs. Thus, labelling of CaM and channels preserved the dominant-negative behavior suggesting apoCaM interaction: preassociated CaM_{MUT}-ECFP seemingly blocks Ca²⁺-sensitive endogenous CaM from accessing the IQ site.

Although the recombinant ECFP and EYFP fusion constructs solved the immediate problem of producing functional and specifically labelled CaM and α_{1C}, there were considerable difficulties measuring steady-state FRET in individual cells. Cell-to-cell variability in the expression of labelled constructs, slow ECFP bleaching, and the inability to selectively excite ECFP excluded many of the popular FRET-detection strategies. To overcome these obstacles, 3³-FRET was used to assay for sensitized EYFP emission, to control for variable ECFP and EYFP expression, as well as to normalize out the inevitable small aberrations of actual optical components in the optical system used to detect FRET.

The principles of 3³-FRET become apparent by considering the fluorescence emission spectrum (FIG. 2A) produced by illuminating a cell expressing both ECFP and EYFP with light at 440 nm. The double-humped shape results from superposition of individual ECFP (thick line) and EYFP (thin line) spectra. FRET alters this spectrum by decreasing the ECFP (energy donor) peak near 480 nm and enhancing the EYFP (energy acceptor) peak near 535 nm. FRET could therefore be nondestructively quantified from the enhanced EYFP emission at 535 nm, but only if it was possible to dissect out EYFP emission secondary to direct excitation (dashed line) from total EYFP emission (thin line) due to both FRET and direct excitation.

As described in detail above, sequential intensity readings were obtained from a single cell using three filter cubes on an epifluorescence microscope, according to the 3³-FRET method.

Control experiments verified that 3³-FRET provides sensitive and selective detection of FRET (FIG. 2B). Averaged data from individual cells expressing only EYFP gave an FR˜1, as expected for this trivial case when no donor is present. Cells co-expressing ECFP and EYFP also showed no FRET, arguing against confounding concentration dependent artifacts such as dimerization or trivial re-absorption. A significant increase in FR was observed for cells expressing a ECFP-EYFP concatemer in which ECFP and EYFP are connected by a 21 amino acid linker. Finally, the genetically-encoded calcium-sensor yellow-cameleon-2 showed the expected Ca²⁺-dependent increase FR.

Two methodological considerations figured importantly in these and subsequent FRET assays: (1) All small, diffusible fluorophores or fluorophore-labelled proteins (such as CaM and ECFP) were expressed with a weak SV40 promoter system rather than standard strong CMV promoters (FIG. 1E); otherwise, recombinant protein concentrations could be high enough to support spurious, concentration-dependent FRET. Expression of channel subunits, which are far less abundant, remained under the control of a CMV promoter to ensure adequate signal levels. (2) Though measurements were collected from entire cells, FRs relating to channels would mostly reflect the interaction of well-folded channels at the surface membrane. This is because channel α_{1C} subunits, tagged intentionally with EYFP, targeted well to the surface membrane (FIG. 1B), and 3³-FRET is based on sensitized EYFP emission.

Armed with this 3³-FRET assay, apoCaM association with L-type channels was investigated (FIG. 3A). Co-expressing ECFP with tagged channel (α_{1C}-EYFP/β₂ₐ/α₂δ) resulted in an FR˜1, ruling out trivial concentration-dependent FRET. In striking contrast, co-expressing CaM_{WT}-ECFP with α_{1C}-EYFP/β₂ₐ/α₂δ supported a marked elevation of FR, indicating that α_{1C}-EYFP and CaM_{WT}-ECFP are in close proximity (<100) in resting cells. Coexpressing CaM_{MUT}-ECFP with labelled channels also caused an elevated FR that was indistinguishable from that observed with CaM_{WT}-ECFP (p˜0.10), arguing strongly that the CaM-channel co-localization in resting cells involves a genuine, Ca²⁺-independent interaction.

One trivial explanation for CaM-channel colocalization would be a generalized enrichment of CaM at the surface membrane, independent of CaM binding to the channel complex. This possibility was excluded by the failure of β₂ₐ-EYFP, which robustly targets the plasma membrane on its own, to support FRET with CaM_{WT}-ECFP in the absence of α_{1C} (FIG. 3B). Moreover, co-expressing CaM_{WT}-ECFP with α_{1C}/β₂ₐ-EYFP/α₂δ restored an elevated FR (FIG. 3B), suggesting that CaM-channel association requires the α_{1C} subunit. The simplest interpretation of these findings is that CaM is an integral subunit of α_{1C}, bound in close proximity to the IQ-like domain through a Ca²⁺-independent interaction with the channel complex.

Like L-type (α_{1C}) channels, P/Q-type (α_{1A}) and R-type (α_{1E}) channel subunits possess homologous IQ-like domains that bind Ca²⁺-CaM in vitro. To test for preassociation of apoCaM to these channel subunits, α_{1E}-EYFP and α_{1A}-EYFP constructs were generated, with carboxyl terminus truncations and EYFP fusions produced as described above.

No form of Ca²⁺-dependent modulation of R-type (α_{1E}) gating has been described thus far. It was surprising, therefore, that co-expressing α_{1E}-EYFP/β₂ₐ/α₂δ with CaM_{MUT}-ECFP supported significant FRET (FIG. 4), providing direct evidence that apoCaM associates with R-type channels. Binding of Ca²⁺-CaM to the IQ-like domain of α_{1A} has recently been unveiled as an essential transduction step in both Ca²⁺-dependent inactivation and facilitation of P/Q-type channels. Cells co-expressing α_{1A}-EYFP/β₂ₐ/α₂δ with CaM_{MUT}-ECFP versus ECFP showed clear elevation of FR (FIG. 4), suggesting that CaM is also a subunit of P/Q-type channels. These results mark the first direct evidence that preassociation of apoCaM is a widely employed strategy among Ca²⁺ channels, and motivates a more extensive search for Ca²⁺-dependent modulation of R-type channels.

FRET not only provides a qualitative indication of whether two tagged protein interact, but in the best cases, it can be used to estimate physical distances between donor and acceptor molecules. However, this estimation requires that each EYFP-tagged molecule be associated with a ECFP-tagged molecule. Since ECFP-tagged moieties (like CaM_{WT}-ECFP) were intentionally limited to avoid trivial concentration-dependent FRET, this condition may not have been satisfied. Using the strategies as discussed above for calculating Equation A23, this limitation was actually turned to advantage. The ECFP and EYFP cube measurements provided the means to estimate the relative concentrations of ECFP- and EYFP-tagged molecules in single cells. When combined with estimation of a single Langmuir binding function, the fraction of EYFP-tagged molecules associated with ECFP-tagged partners can be calculated and the calculated fraction can be used to predict FR according to Equation A23.

FIG. 5A shows the application of such analysis to the pairing of α_{1C}-YFP and CaM_{WT}-CFP. The upper FR-A_{b} plot indicates a robust fit of the binding model to data, with FR rising from 1 at A_{b}˜0 toward an FRₘₐₓ of 2.9 at A_{b}=1. Shown below are the distributions of the relative numbers of CaM_{WT}-CFP and α_{1C}-YFP molecules (N_{D} and N_{A}, respectively) and the corresponding molar expression ratio of CaM_{WT}-CFP to α_{1C}-YFP molecules (N_{D}/N_{A}). The large cell-to-cell variability of this molar expression ratio ensured exploration of nearly the full range of fractional occupancies (A_{b}). By contrast, control cells coexpressing CFP and (α_{1C}-YFP (FIG. 5B) give rise to clustering of FR-A_{b} data at A_{b}˜0 with an FR˜1 despite a similar 25-fold distribution of N_{D}/N_{A} ratios. Hence, the wide variation of molar expression ratios of α_{1C}-YFP and CaM_{WT}-CFP would not, in itself, cause artifactual elevation of FR above unity. Another revealing case involves cells expressing yellow-cameleon-2 (FIG. 5C), for which the FR data congregated at A_{b}˜1, as expected for a molecule incorporating both CFP and YFP in a fixed 1:1 stoichiometry. This clustering at A_{b}˜1 further supported the accuracy of the A_{b} estimations produced by our model. Interestingly, the N_{A}/N_{D} ratios for yellow-cameleon-2 are concentrated at ˜1, arguing strongly that estimates of relative N_{A} and N_{D} in our model are related by a single constant of proportionality to the actual numbers of acceptor and donor molecules.

We extended this analysis to all of our FRET pairs. A summary table of the parameters resulting from these fits is shown in FIG. 5D. FRₘₐₓ values for α_{1C}-YFP coexpressed with CaM_{WT}-CFP matched those for α_{1C}-YFP with CaM_{MUT}-CFP, further emphasizing that the detected association entails an authentic Ca²⁺-independent interaction. Interestingly, whereas FRₘₐₓ values for the different channels were all ˜3 (equal to the FR measured for Ca²⁺-free yellow-cameleon-2), K_{d,EFF} varied substantially. This suggests that the relative affinities for apoCaM are different while the binding sites are positioned similarly. FRₘₐₓ values corresponding to association of β₂ₐ with CaM_{WT} and α_{1C} with β₂ₐ were similar (FIG. 5D-E). In the case of FRET between labelled α_{1C} and β₂ₐ a subunits, measured FRs were predominantly equal to FRₘₐₓ (FIG. 5E), fitting with previous findings that membrane targeting of α_{1C} requires β₂ₐ association (Bichet et al., 2000).

Finally, determination of FRₘₐₓ values enabled initial estimates of relative inter-fluorophore distances (see Procedures). This formed the basis for the triangle in FIG. 5F, which proposes the relative arrangement of key landmarks on the cytoplasmic aspect of the channel: the auxiliary β₂ₐ subunit, the α_{1C} carboxyl tail just distal to the IQ site, and preassociated CaM. Labelled CaM and α_{1C} supported an FRₘₐₓ of ˜3, which corresponds to an inter-fluorophore distance of approximately 60 Å provided that it is assumed that the interfluorophore orientations are sufficiently randomized. The pairing of labelled β₂ₐ with either labelled CaM_{WT} or α_{1C} yielded the same FRₘₐₓ of 1.2, corresponding to a comparatively larger inter-fluorophore distance of about 90 Å. Although there are critical caveats to such distance calculations (see Erickson, et al., 2001, Neuron 31:973-985 for complete discussion), it is interesting to consider the relative dimensions of the triangle. For example, although changes in R₀ can arise from differences in interfluorophore orientations, the magnitude of such changes of R₀, observed over the majority of possible orientations, results in less than 20-30% variation in predicted distances. In favorable instances, these dimensions may prove useful in establishing first-order physical constraints on the organization of a Ca²⁺ channel complex.

### Example 2

### Application of 3³-FRET for Two-Hybrid Mapping of the Molecular Contacts Underlying Ca²⁺-Dependent Moldulation of L-type Ca²⁺ Channels

Based on the work presented in Example 1, application of the 3³-FRET method to fluorophore tagged Ca²⁺ channels and calmodulin (CaM, the Ca²⁺ sensor for channel modulation) revealed that these two proteins bind in resting cells, and that the association does not require Ca²⁺. This raises two fundamental questions. Where exactly does Ca²⁺-free CaM (apoCaM) bind to the channel? And, how is Ca²⁺-activation of preassociated apoCaM coupled to modulation of channel gating?

3³-FRET is uniquely equipped to answer these questions, in particular because of its ability to compare FRₘₐₓ and K_{d,EFF} among different FRET pairs. 3³-FRET was therefore applied for single cell, two-hybrid screening of channel/CaM interactions, with ECFP-tagged CaM serving as “bait” and EYFP-tagged segments of the Ca²⁺ channel as “prey.”

The first objective was to identify which Ca²⁺ channel segments coordinate binding of apoCaM. A library of short (˜100 basepair, or ˜33 residue) and long (˜200 basepair, or ˜66 residue) segments from the L-type Ca²⁺ channel carboxyl tail was generated, and each segment was fused in frame to EYFP. Four such segments are illustrated in FIG. 7A (EF, PreIQ, IQ and PreIQ-IQ). The EYFP-tagged segments were then cotransfected in cells with CaM_{MUT}-CFP, which incorporates the Ca²⁺ insensitive mutant CaM, and the cells where probed with 3³-FRET.

No interaction was detected between EF-YFP and CaM_{MUT}-CFP, based on an FR˜1 (FIG. 7B). Although PreIQ-YFP or IQYFP individually supported only weak to moderate FRET signals with CaM_{MUT}-CFP, a segment containing both PreIQ and IQ sustained robust FRET with FR˜2. However, it is essential to determine whether these disparate FRET readings are due merely to different donor/acceptor orientations (FRₘₐₓ) or, more importantly, different binding affinities (K_{d,EFF}). Preliminary results from application of 3³-FRET revealed that despite having similar FRₘₐₓ values, the combined PreIQ-IQ segment supported the lowest K_{d,EFF} (FIG. 7B, right), suggesting that PreIQ and IQ each contribute to the formation of a high-affinity apoCaM binding pocket. This could explain the lack of agreement among earlier in vitro tests of preassociation (see, for discussion, Erickson, et al., 2001, Neuron 31: 973-985), as a tertiary binding structure may be especially vulnerable to solubilizing conditions.

The 3³-FRET method was also applied to investigate how Ca²⁺-activation of preassociated CaM could trigger channel modulation. The cells were clamped to either high (10 mM) or low (5 mM EGTA) internal Ca²⁺ before application of 3³-FRET. Both PreIQ/CaM and IQ/CaM exhibited marked conformational changes upon elevation of intracellular Ca²⁺, based on dramatic increases in FRₘₐₓ (FIG. 7C, right; compare black and gray arrowheads). Monitoring these Ca²⁺-induced changes in FR provides an exciting vantage into the molecular movements underlying Ca²⁺-dependent modulation.

As a 2-hybrid screening assay, 3³-FRET generally exhibits a very low false-positive rate, as FRET signals are only detected when the donor and acceptor fluorophores are within 100 Å. However, the false-negative rate can be high, since the orientation and/or distance of the fluorophores tagging two polypeptides might not be conducive to FRET, even when the two polypeptides are tightly bound to one another. Thus 3³-FRET based two-hybrid screening compliments existing hybridization assays that exhibit high false-positive rates, such as yeast two-hybrid screening. Having complimentary screening assays is advantageous, since the objectives for a particular screen can be matched with the assay that offers the best trade-off between high false-positives or high false-negatives. Moreover, 3³-FRET provides the unique ability to determine the binding affinity for polypeptides that do interact, which enables discrimination between strong and weak interactions.

Variations, modifications, and other implementations of what is described herein will occur to those of ordinary skill in the art without departing from the spirit and scope of the invention. All publications, patents, patent applications and references cited herein and in the provisional application to which this application claims priority are incorporated by reference in their entireties.

What is claimed is:

1. A method for detecting a FRET signal generated by an interaction between a donor and acceptor molecule in a sample, comprising:
(a) determining the ratio of contribution of total acceptor emission at the emission wavelength of the acceptor to the contribution of acceptor emission at the same wavelength due to direct excitation only; and
(b) correlating the ration with the physical distance between a donor:acceptor pair, thereby providing a measure of a FRET signal.

2. The method according to claim 1, further comprising obtaining sequential light intensity readings from the sample.

3. The method according to claim 1, wherein the donor molecule is CFP, ECFP, or GFP.

4. The method according to claim 1 or 3, wherein the acceptor molecule is YFP, EYFP, or dsRed.

5. The method according to claim 1, wherein the strength of FRET is specified by the FRET ratio, processed according to:
$$FR = \frac{[ {S}_{FRET} ( DA ) - {R}_{D1} {S}_{D} ( DA ) ]}{{R}_{A1} [ {S}_{A} ( DA ) - {R}_{D2} {S}_{D} ( DA ) ]}$$
wherein S_{FRET}(DA) is a measure of light intensity transmitted to the detector from the third filter, S_{D}(DA) is a measure of light intensity transmitted to the detector from first filter, and S_{A}(DA) is a measure of light intensity transmitted to the detector from second filter,
wherein R_{D1}, R_{A1}, and R_{D2} are predetermined constants determined from measurements of light emissions from specimens expressing only donor or acceptor molecules.

6. The method according to claim 5, further comprising the step of determining FRET efficiency (E) by solving for E using the formula
E=(FR−1)[ε_{A}(λex)/ε_{D}(λex)],
wherein the bracketed term is the ratio of acceptor and donor molar extinction coefficients scaled for the third filter.

7. The method according to claim 6, further comprising the step of determining donor:acceptor distance using the formula;
R=R₀(E⁻¹−1)^{1/6},
wherein R₀=49.

8. The method according to claim 1, further comprising step of determining the fraction of acceptor molecules associated with donor molecules.

9. The method according to claim 2, wherein the sequential light intensity readings are obtained using three filter cubes, each filter cube comprises an excitation filter, a dichroic mirror, and an emission filter.

10. The method according to claim 1, wherein the method further comprises providing an optical system comprising:
(i) a light source for providing excitation light to the specimen;
(ii) a detector;
(iii) a specimen holder for positioning the specimen in a suitable position to receive light from the light source sufficient to excite the donor; and to transmit light emitted by the cell to the detector; and
(iv) a holder for sequentially receiving a first, second, and third filter, and for positioning each of the filters, sequentially, in the light path from the specimen to detector.

11. The method according to claim 1, wherein the specimen is a cell.

12. The method according to claim 11, further comprising the step of introducing the donor and acceptor molecule into the cell.

13. The method according to claim 12, wherein introducing fluorophores is performed by cDNA transfection, transformation, electroporation, microinjection, or a combination thereof.

14. The method according to claim 1, wherein the donor molecule and acceptor molecule are each linked to different biomolecules.

15. The method according to claim 14, wherein the different biomolecules are binding partners.

16. The method according to claim 15, wherein the different biomolecules are different polypeptides.

17. The method according to claim 1, wherein the donor molecule and acceptor molecule are linked to a single molecule for detecting an analyte.

18. The method according to claim 17, wherein the molecule for detecting an analyte specifically binds to the analyte.

19. The method according to claim 18, wherein the molecule for detecting an analyte is cleavable by the analyte.

20. The method according to claim 14, wherein the donor molecule and acceptor molecules comprise polypeptides.

21. The method according to claim 20, wherein the donor molecule and acceptor molecules are fused in frame to the polypeptides.

22. The method according to claim 14, wherein at least one of the different biomolecules comprises a polynucleotide.

23. The method according to claim 17, wherein the molecule for detecting an analyte comprises a polypeptide.

24. The method according to claim 17, wherein the molecule for detecting an analyte comprises a polynucleotide.

25. The method according to claim 20, wherein one of the polypeptides is selected from the group consisting of calmodulin (CaM), cGMP-dependent protein kinase, a steroid hormone receptor or a ligand binding domain thereof, protein kinase C, inositol-1,4,5-triphosphate receptor, alphachymotrypsin, or recoverin.

26. The method according to claim 20, wherein one of the polypeptides comprises a protease cleavage site.

27. The method according to claim 20, wherein one or both of the polypeptides comprises an intracellular localization signal for localizing one or both of the polypeptides into a cell.

28. The method according to claim 17, wherein the molecule for detecting an analyte is immobilized on a solid phase, thereby forming a FRET sensor.

29. The method according to claim 28, further comprising exposing the FRET sensor to a sample suspected of comprising the analyte.

30. The method according to claim 29, wherein the measure of FRET is correlated with the presence or level of the analyte.

31. The method according to claim 12, wherein the donor molecule and acceptor molecule are linked to a single molecule for detecting an analyte, and wherein the measure of FRET is correlated with the presence or level of analyte in the cell.

32. The method according to claim 12, wherein the donor molecule and acceptor molecule are each linked to a different biomolecule.

33. The method according to claim 32, wherein the different biomolecules are binding partners and the measure of FRET is correlated to binding of the binding partners to each other.

34. The method according to claim 32, further comprising:
exposing the cell to a sample suspected of comprising a modulator of binding of the binding partners and wherein the measure of FRET indicates whether or not the sample comprises the modulator.

35. The method according to claim 33, wherein one of the binding partners is an intracellular signaling molecule.

36. The method according to claim 33, wherein the binding partners are selected from the group consisting of: a ligand and receptor; antibodies and antigens; calmodulin and calcium; and GTP and G-Coupled Protein Receptors.

37. The method according to claim 33, further comprising the step of contacting the cell with a compound, and measuring a change in FRET at a first time and at a second time.

38. The method according to claim 1, wherein the donor molecule is linked to a bait polypeptide, and wherein the acceptor molecule is linked to a prey polypeptide, and wherein the measure of FRET provides a measure of whether the bait polypeptide and prey polypeptide specifically bind to each other.

39. The method according to claim 12, further comprising the step of sorting cells comprising donor and acceptor molecules from cells which do not comprise donor acceptor molecules.

40. The method according to claim 39, comprising the step of sorting cells wherein donor and acceptor molecules are in sufficient proximity to exhibit FRET.

41. The method according to claim 1, wherein a donor and acceptor pair are selected from the list of fluorophores shown in Table 1.

42. The method according to 38, further comprising the step of performing FRET detection for a plurality of different prey polypeptides.

43. The method according to claim 42, wherein FRET detection is performed using a plate reader.

44. The method according to claim 38 or 42, wherein when FRET is detected between a donor molecule linked to a bait polypeptide, and an acceptor molecule linked to a prey polypeptide, the sequence of said prey polypeptide is determined.

45. The method according to claim 38, wherein said bait and prey polypeptides are expressed in a cell.

46. The method according to claim 45, wherein when FRET is detected, the cell is lysed.

47. The method according to claim 43, wherein said plate reader is coupled to a robotic fluid transfer system.

48. The method according to claim 33, wherein one or more of the binding partners comprises one or more mutations.

49. A method for determining FRET between a donor-tagged molecule and an acceptor-tagged molecule comprising determining a maximum FRET ratio where every acceptor-tagged molecule is associated with a donor-tagged molecule and minimizing the value (FRₑₓₚ-FR_{predicted})².

50. A computer program product for implementing the steps shown in FIG. 8B.
